(19) United States
(12) Patent Application Publication
(10) Pub. No.: US 2026/0003429 A1
(43) Pub. Date: Jan. 1, 2026

(54) DEVICES, SYSTEMS, AND METHODS FOR CREATING AND MANAGING HEALTH RECORDS USING DATA GENERATED BY FLEXIBLE CIRCUITS

(71) Applicant: Liquid Wire Inc., Portland, OR (US)

(72) Inventors: Mark William Ronay, Portland, OR (US); Jon Carl Ohrn, Chatham, NJ (US); Jorge E. Carbo, JR., Portland, OR (US); Trevor Antonio Rivera, Portland, OR (US); Michael Adventure Hopkins, Portland, OR (US); Katherine M. Nelson, Portland, OR (US); Casey Culbertson, West Linn, OR (US); Iheanyi Eronini Marike, Portland, OR (US); Jerry Gene Sewell, JR., Portland, OR (US); Jesse Michael Martinez, Milwaukie, OR (US); Michael Jasper Wallans, Portland, OR (US)

(73) Assignee: Liquid Wire Inc., Portland, OR (US)

(21) Appl. No.: 19/045,395

(22) Filed: Feb. 4, 2025

Related U.S. Application Data
(63) Continuation of application No. PCT/US23/71792, filed on Aug. 7, 2023.
(60) Provisional application No. 63/370,615, filed on Aug. 5, 2022.

(51) Int. Cl. G06F 3/01; G06F 1/16; G16H 10/60; H04L 9/00; H05K 1/03

(52) U.S. Cl. CPC G06F 3/014; G06F 1/163; G06F 3/017; G16H 10/60; H05K 1/0393; H04L 9/50; H05K 2201/0382

(57) ABSTRACT

A computer-implemented method of autonomously dispositioning data generated by a wearable article in compliance with multiple application-specific requirements is disclosed herein. The method can include predefining one or more rules by which data generated by a wearable article should be managed, wherein the one or more rules include definition of a triggering event. The method can further include receiving data associated with motions of the wearable article, wherein the data includes information associated with electrical parameters generated by the wearable article that vary with the motions of the wearable article. The method can further include detecting an initiation of the triggering event and managing subsequent data generated by the wearable article, including data associated with varying electrical parameters, in accordance with the predefined one or more rules.

### CROSS-REFERENCE TO RELATED APPLICATIONS

The present application is a continuation of and claims the benefit of priority from PCT/US2023/071792, filed on Aug. 7, 2023, which claims the benefit of priority from U.S. Provisional Patent Application No. 63/370,615, titled DEVICES, SYSTEMS, AND METHODS FOR CREATING AND MANAGING HEALTH RECORDS USING DATA GENERATED BY FLEXIBLE CIRCUITS, filed Aug. 5, 2022. The disclosures of both applications are herein incorporated by reference in their entireties.

### FIELD

The present disclosure is generally related to flexible circuits and, more particularly, is directed to flexible circuits that can be either integrated into wearable articles for the purposes of generating data which can be minted into a non-fungible token that can be used to simulate motions in a virtual environment that correspond to physical motions in a real environment.

### SUMMARY

The following summary is provided to facilitate an understanding of some of the innovative features unique to the aspects disclosed herein and is not intended to be a full description. A full appreciation of the various aspects can be gained by taking the entire specification, claims, and abstract as a whole.

In various aspects, a computer-implemented method of autonomously dispositioning data generated by a wearable article in compliance with multiple application-specific requirements is disclosed. The method can include predefining, via a processor, one or more rules by which data generated by a wearable article should be managed, wherein the one or more rules include definition of a triggering event, receiving, via the processor, data associated with motions of the wearable article, wherein the data includes information associated with electrical parameters generated by the wearable article that vary with the motions of the wearable article, detecting, via the processor, an initiation of the triggering event, and managing, via the processor, subsequent data generated by the wearable article, including data associated with varying electrical parameters, in accordance with the predefined one or more rules.

In various aspects, a system is disclosed. The system can include a wearable article including a flexible circuit, wherein the flexible circuit includes a trace made from a deformable conductor configured to generate varying electrical parameters in response to motions of the wearable article, and a computing device communicably coupled to the wearable article, wherein the computing device includes a processor and a memory configured to store instructions that, when executed by the processor, cause the computing device to predefine one or more rules by which data generated by a wearable article should be managed based on a user input, wherein the one or more rules include definition of a triggering event, receive data associated with motions of the wearable article, wherein the data includes information associated with the varying electrical parameters generated by the deformable conductor, detect an initiation of the triggering event, and manage subsequent data generated by the wearable article, including data associated with varying electrical parameters, in accordance with the predefined one or more rules.

In various aspects, a method of managing health records using a wearable article comprising a flexible circuit is disclosed. The method can include generating, via the wearable article, a plurality of data entries, wherein each data entry of the plurality includes a key component including searchable metadata and a value component associated with electrical parameters generated by a deformable conductor of the flexible circuit, detecting, via a computing device, a subset of confidential data entries based on the key component of each data entry of the subset, and storing, via the computing device, the subset of data entries in a confidential storage, wherein the confidential storage complies with a regulation governing the management of confidential health records.

These and other features and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

Various features of the aspects described herein are set forth with particularity in the appended claims. The various aspects, however, both as to organization and methods of operation, together with advantages thereof, may be understood in accordance with the following description taken in conjunction with the accompanying drawings as follows:

FIG. 1 illustrates a system configured to generate and store data associated with the motions of a wearable article featuring flexible circuits, including a blockchain network and/or other data repositories, in accordance with at least one non-limiting aspect of the present disclosure;

FIG. 2 illustrates a block diagram of a system for implementing a blockchain network configured to host an NFT, in accordance with at least one non-limiting aspect of the present disclosure;

FIG. 3 illustrates a method of autonomously dispositioning data generated by a single wearable article in compliance with multiple application-specific requirements, in accordance with at least one non-limiting aspect of the present disclosure;

FIGS. 4A, and 4B, and 4C illustrate a wearable article configured for use with the system of FIG. 1, in accordance with at least one non-limiting aspect of the present disclosure;

FIGS. 5A-D illustrate another wearable article configured for use with the system of FIG. 1, in accordance with at least one non-limiting aspect of the present disclosure; and

FIG. 6 illustrates a table of data generated by a wearable article featuring flexible circuits, in accordance with at least one non-limiting aspect of the present disclosure;

FIG. 7 illustrates a means of indexing the data entries of the table of FIG. 6, in accordance with at least one non-limiting aspect of the present disclosure;

FIG. 8 illustrates a method of managing health records using a wearable article comprising a flexible circuit, in accordance with at least one non-limiting aspect of the present disclosure; and

FIG. 9 illustrates an architecture that can be deployed via the system of FIG. 1 to generate and store data associated with the motions of a wearable article featuring flexible circuits, in accordance with at least one non-limiting aspect of the present disclosure.

Corresponding reference characters indicate corresponding parts throughout the several views. The exemplifications set out herein illustrate various aspects of the invention, in one form, and such exemplifications are not to be construed as limiting the scope of the invention in any manner.

### DETAILED DESCRIPTION

Numerous specific details are set forth to provide a thorough understanding of the overall structure, function, manufacture, and use of the aspects as described in the disclosure and illustrated in the accompanying drawings. Well-known operations, components, and elements have not been described in detail so as not to obscure the aspects described in the specification. The reader will understand that the aspects described and illustrated herein are non-limiting examples, and thus it can be appreciated that the specific structural and functional details disclosed herein may be representative and illustrative. Variations and changes thereto may be made without departing from the scope of the claims. Furthermore, it is to be understood that such terms as “forward”, “rearward”, “left”, “right”, “upwardly”, “downwardly”, and the like are words of convenience and are not to be construed as limiting terms. Furthermore, it is to be understood that such terms as “forward”, “rearward”, “left”, “right”, “upwardly”, “downwardly”, and the like are words of convenience and are not to be construed as limiting terms. In the following description, like reference characters designate like or corresponding parts throughout the several views of the drawings. Also in the following description, it is to be understood that such terms as “forward”, “rearward”, “left”, “right”, “upwardly”, “downwardly”, and the like are words of convenience and are not to be construed as limiting terms.

A portion of the disclosure of this patent document contains material which is subject to copyright protection. The copyright owner has no objection to the facsimile reproduction by anyone of the patent disclosure, as it appears in the Patent and Trademark Office patent files or records, but otherwise reserves any and all copyrights disclosed herein.

Electronic circuits that are flexible and deformable have emerged as a means of innovating conventional electronics and introducing electronics into new products and applications. However, most flexible circuits are limited in how much they can be deformed prior to fatiguing and failing. That said, a change in circuit geometry could lead to a subsequent change in electrical parameters generated across a flexible circuit, which could be used to characterize a structural parameter or condition of the circuit, as desired. Thus, it is conceivable that deformable conductors can be implemented in wearable articles such that electrical parameters can be generated and subsequently correlated to physical motions, which can be used to characterize a performance given by an athlete, an artist, a celebrity, a politician, a teacher, or any other person of interest. However, it shall be appreciated that the expansive number of applications for such electronics will necessitate the ability to separately and uniquely handle information associated with such flexible circuits in different ways.

For example, flexible circuits may be incorporated into wearable articles configured for recreational and/or personal use, in which case the user may want to use information associated with the flexible circuits however they see fit, without restriction or regulation. The average healthcare consumer, for example, might utilize a wearable article with flexible circuits for athletic purposes, to monitor, track, and characterize their performance. However, some wearable articles can implement flexible circuits to generate electrical parameters that can be correlated to the supervised and/or prescribed health of a user. For example, the average healthcare consumer may also utilize a wearable article with flexible circuits for medical and/or rehabilitative purposes. It is important that data generated by the wearable article with flexible circuits can be marked, indexed, and segmented for confidential purposes and/or applications, in compliance with laws and regulations, such as the Health Insurance Portability and Accountability Act of 1996 (“HIPAA”). Alternately, an insurance company may want to access and review information associated with the flexible circuits as a condition of their coverage.

In other words, how information associated with flexible circuits is handled by a system upon generation is important, and dependent on the particular application of the technology. Conventional devices, systems, and methods, for example, may necessitate that a user have different wearable articles for different applications, based on the aforementioned concerns. Accordingly, there is a need for devices, systems, and methods for creating and managing health records that include data generated by flexible circuits, via NFTs and other confidential storage media.

While certain electronic components typically have some inherent flexibility, that flexibility is typically constrained both in the amount the components can flex, their resilience in flexing, and the number of times the electronic components can flex before the electronic components deteriorate or break. Consequently, the utility of such electronic components in various environments may be limited, either by reliability or longevity or by the ability to function at all. Moreover, the lateral size of such components may result in additional stresses placed on the component.

The use of conductive gel, however, provides for electronic components that are flexible and deformable while maintaining resiliency. Moreover, the operational flexing, stretching, deforming, or other physical manipulation of a conductive trace formed from conductive gel may produce predictable, measurable changes in the electrical characteristics of the trace. By measuring the change in resistance or impedance of such a trace the change in length of the trace may be inferred. By combining the changes in lengths of multiple traces, the relative movement of points on a two-dimensional surface may be calculated.

As previously described, according to some non-limiting aspects, it might be beneficial to store data generated by a wearable article featuring flexible circuits in an NFT for health-related applications. For example, as previously discussed, data can be generated by a wearable article featuring flexible circuits and thus, that data can be associated with electrical the motions of a user while wearing the wearable article. However, according to some non-limiting aspects, the user may not be a performer but an average healthcare consumer. The average healthcare consumer may utilize wearable articles with flexible circuits in a variety of different situations.

For example, the average healthcare consumer might utilize a wearable article with flexible circuits for athletic purposes, to monitor, track, and characterize their performance in ways similar to those previously discussed. The average healthcare consumer, however, may also utilize a wearable article with flexible circuits for medical and/or rehabilitative purposes. Thus, it is important that data generated by the wearable article with flexible circuits can be marked, indexed, and segmented for confidential purposes and/or applications, in compliance with laws and regulations, such as the Health Insurance Portability and Accountability Act of 1996 (“HIPAA”). Accordingly, there is a need for devices, systems, and methods for creating and managing health records using NFTs and data generated by flexible circuits.

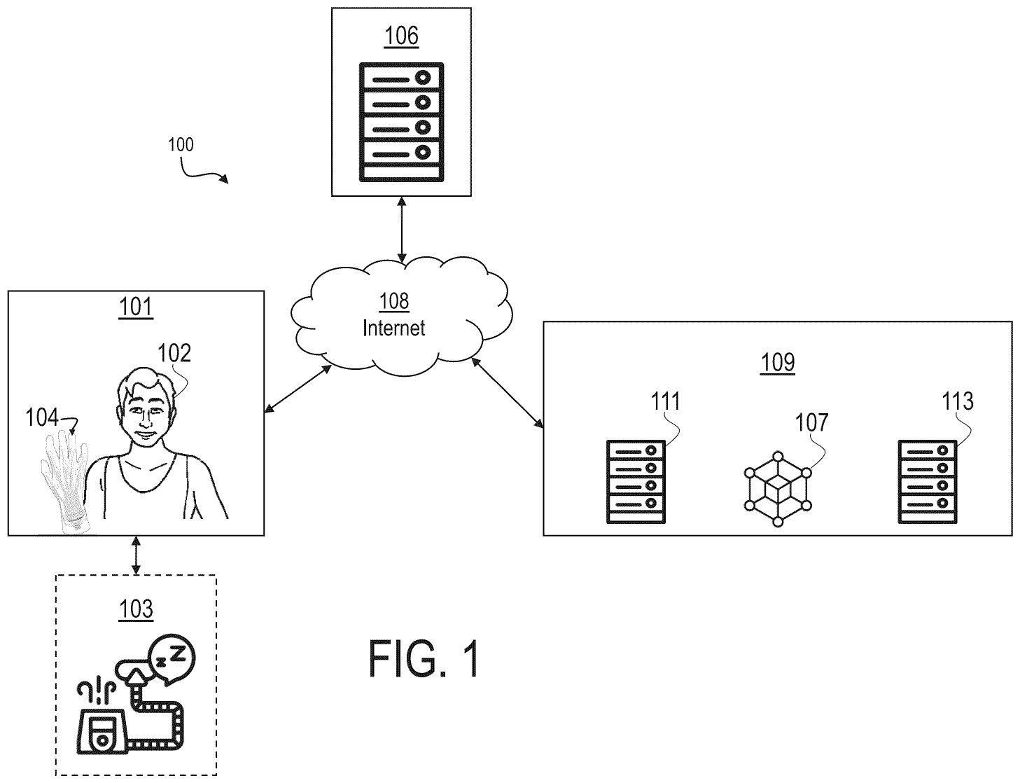

Referring now to FIG. 1, a system 100 configured to generate and store data associated with the motions of a wearable article 104 featuring flexible circuits, is depicted in accordance with at least one non-limiting aspect of the present disclosure. As will be described herein, the system 100 can manage data generated by the wearable article 104 can be stored in one or more data repositories 109, such as a personal server 111, a blockchain network 107, and/or a third-party server 113, amongst other data repositories, any of which can be secured in compliance with user preference and/or governing laws or regulations. For example, according to some non-limiting aspects, the personal server 111 can be a private repository owned by the user 102, secured by credentials (e.g., username, password, biometrics, etc.), the blockchain network 107 can be cryptographically secured, and the third-party server can be secured via a third-party, such as an insurer of the user 102, for example. Of course, the aforementioned examples are merely illustrative and not intended to be limited, as the one or more repositories 109 can include any number of data stores controlled by an entity via any means.

According to the non-limiting aspect of FIG. 1, the system 100 can include a user 102 wearing a wearable article 104 featuring flexible circuits in a physical environment 101. The system can further include a computing device 106 and a blockchain network 107, wherein the wearable article 104 can be communicably coupled to the computing device 106, and the one or more data repositories 109. For example, the wearable article 104, the computing device 106, and the one or more data repositories 109 can be communicably coupled via the internet 108 by any means of wireless and/or wired connection. For example, according to some non-limiting aspects, the wearable article 104, the computing device 106, and the one or more data repositories 109 can be communicably coupled via a wireless access point. However, according to other non-limiting aspects, at least the wearable article 104 can include a local memory device and can be configured to be connected to the computing device 106 via a wired connection, such that time-stamped data generated by the flexible circuits and stored in the local memory can be transmitted to the computing device 106.

According to the non-limiting aspect of FIG. 1, the wearable article 104 can include a glove worn on a hand of the user. For example, the glove 104 of FIG. 1 can be similarly configured to any of the wearable articles disclosed in U.S. Provisional Application No. 63/268,063, titled DEVICES, SYSTEMS, AND METHODS FOR GENERATING AND CORRELATING ELECTRICAL PARAMETERS TO THE PHYSICAL MOTIONS OF A USER, filed Feb. 15, 2022, or U.S. Provisional Application No. 63/368,140, titled DEVICES, SYSTEMS, AND METHODS FOR SIMULATING MOTIONS IN A VIRTUAL ENVIRONMENT VIA WEARABLE ARTICLES WITH FLEXIBLE CIRCUITS, filed Jul. 11, 2022, the disclosures of which is hereby incorporated by reference in its entirety. However, it shall be appreciated that the system 100 can utilize any type of wearable article that features flexible circuits made from deformable conductors, such as those described in International Patent Application No. PCT/US2017/019762 titled LIQUID WIRE, which was filed on Feb. 27, 2017 and published on Sep. 8, 2017 as International Patent Publication No. WO2017/151523A1.

For example, each trace of the wearable article 104 can include a variety of forms, such as a liquid, a paste, a gel, and/or a powder, amongst others that would enable the traces 104a, 104b to have a deformable (e.g., soft, flexible, stretchable, bendable, elastic, flowable viscoelastic, Newtonian, non-Newtonian, etc.) quality. According to some non-limiting aspects, the deformable, conductive materials can include an electroactive material, such as a deformable conductors produced from a conductive gel (e.g., a gallium indium alloy). The conductive gel can have a shear thinning composition and, according to some non-limiting aspects, can include a mixture of materials in a desired ratio. For example, according to one preferable non-limiting aspect, the conductive gel can include a weight percentage of a eutectic gallium alloy between 59.9% and 99.9% and a weight percentage of a gallium oxide between 0.1% and about 2.0%. Of course, the present disclosure contemplates other non-limiting aspects, featuring traces of varying forms and/or compositions to achieve the benefits disclosed herein.

For example, the wearable article 104 can include flexible circuits with traces formed from a deformable conductive material that is optimized to have a viscosity such that the deformable conductive material is able to heal upon unitization of the layers but not such that the deformable conductive material overly deforms and does not achieve the intended pattern. As another example, adhesive characteristics and/or viscosity of the deformable conductive material may be optimized such that it remains on the substrate layer upon removal of the removable stencil 50 and but does not adhere to the channels 504, 506 of the stencil thereby lifting the deformable conductive material off of the substrate layer. In some aspects, a viscosity of the deformable conductive material may, when under high shear (e.g., in motion), be in a range of about 10 Pascal seconds (Pa*s) and 500 Pa*s, such as a range of 50 Pa*s and 300 Pa*s, and/or may be about 50 Pa*s, about 60 Pa*s, about 70 Pa*s, about 80 Pa*s, about 90 Pa*s, about 100 Pa*s, about 110 Pa*s, about 120 Pa*s, about 130 Pa*s, about 140 Pa*s, about 150 Pa*s, about 160 Pa*s, about 170 Pa*s, about 180 Pa*s, about 190 Pa*s, or about 200 Pa*s. In some aspects, a viscosity of the deformable conductive material may, when under low shear (e.g., at rest), be in a range of 1,000,000 Pa*s and 40,000,000 Pa*s and/or may be about 10,000,000 Pa*s, about 20,000,000 Pa*s, about 30,000,000 Pa*s, or about 40,000,000 Pa*s.

The electrically conductive compositions can comprise a mixture of a eutectic gallium alloy and gallium oxide, wherein the mixture of eutectic gallium alloy and gallium oxide has a weight percentage (wt %) of between about 59.9% and about 99.9% eutectic gallium alloy, such as between about 67% and about 90%, and a wt % of between about 0.1% and about 2.0% gallium oxide such as between about 0.2 and about 1%. For example, the electrically conductive compositions can have about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or greater, such as about 99.9% eutectic gallium alloy, and about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, and about 2.0% gallium oxide.

The eutectic gallium alloy can include gallium-indium or gallium-indium-tin in any ratio of elements. For example, a eutectic gallium alloy includes gallium and indium. The electrically conductive compositions can have any suitable percentage of gallium by weight in the gallium-indium alloy that is between about 40% and about 95%, such as about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, or about 95%.

The electrically conductive compositions can have a percentage of indium by weight in the gallium-indium alloy that is between about 5% and about 60%, such as about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, or about 60%.

The eutectic gallium alloy can include gallium and tin. For example, the electrically conductive compositions can have a percentage of tin by weight in the alloy that is between about 0.001% and about 50%, such as about 0.001%, about 0.005%, about 0.01%, about 0.05%, about 0.1%, about 0.2%, about 0.3%, about 0.4%, about 0.5%, about 0.6%, about 0.7%, about 0.8%, about 0.9%, about 1%, about 1.5%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50%.

The electrically conductive compositions can comprise one or more micro-particles or sub-micron scale particles blended with the eutectic gallium alloy and gallium oxide. The particles can be suspended, either coated in eutectic gallium alloy or gallium and encapsulated in gallium oxide or not coated in the previous manner, within eutectic gallium alloy. The micro-or sub-micron scale particles can range in size from nanometer to micrometer and can be suspended in gallium, gallium-indium alloy, or gallium-indium-tin alloy. Particle to alloy ratio can vary and can change the flow properties of the electrically conductive compositions. The micro and nanostructures can be blended within the electrically conductive compositions through sonication or other suitable means. The electrically conductive compositions can include a colloidal suspension of micro and nanostructures within the eutectic gallium alloy/gallium oxide mixture.

The electrically conductive compositions can further include one or more micro-particles or sub-micron scale particles dispersed within the compositions. This can be achieved in any suitable way, including by suspending particles, either coated in eutectic gallium alloy or gallium and encapsulated in gallium oxide or not coated in the previous manner, within the electrically conductive compositions or, specifically, within the eutectic gallium alloy fluid. These particles can range in size from nanometer to micrometer and can be suspended in gallium, gallium-indium alloy, or gallium-indium-tin alloy. Particle to alloy ratio can vary, in order to, among other things, change fluid properties of at least one of the alloys and the electrically conductive compositions. In addition, the addition of any ancillary material to colloidal suspension or eutectic gallium alloy in order to, among other things, enhance or modify its physical, electrical or thermal properties. The distribution of micro and nanostructures within the at least one of the eutectic gallium alloy and the electrically conductive compositions can be achieved through any suitable means, including sonication or other mechanical means without the addition of particles. In certain embodiments, the one or more micro-particles or sub-micron particles are blended with the at least one of the eutectic gallium alloy and the electrically conductive compositions with wt % of between about 0.001% and about 40.0% of micro-particles, for example about 0.001%, about 0.005%, about 0.01%, about 0.05%, about 0.1%, about 0.2%, about 0.3%, about 0.4%, about 0.5%, about 0.6%, about 0.7%, about 0.8%, about 0.9%, about 1%, about 1.5%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40.

The one or more micro- or sub-micron particles can be made of any suitable material including soda glass, silica, borosilicate glass, quartz, oxidized copper, silver coated copper, non-oxidized copper, tungsten, super saturated tin granules, glass, graphite, silver coated copper, such as silver coated copper spheres, and silver coated copper flakes, copper flakes, or copper spheres, or a combination thereof, or any other material that can be wetted by the at least one of the eutectic gallium alloy and the electrically conductive compositions. The one or more micro-particles or sub-micron scale particles can have any suitable shape, including the shape of spheroids, rods, tubes, a flakes, plates, cubes, prismatic, pyramidal, cages, and dendrimers. The one or more micro-particles or sub-micron scale particles can have any suitable size, including a size range of about 0.5 microns to about 60 microns, as about 0.5 microns, about 0.6 microns, about 0.7 microns, about 0.8 microns, about 0.9 microns, about 1 microns, about 1.5 microns, about 2 microns, about 3 microns, about 4 microns, about 5 microns, about 6 microns, about 7 microns, about 8 microns, about 9 microns, about 10 microns, about 11 microns, about 12 microns, about 13 microns, about 14 microns, about 15 microns, about 16 microns, about 17 microns, about 18 microns, about 19 microns, about 20 microns, about 21 microns, about 22 microns, about 23 microns, about 24 microns, about 25 microns, about 26 microns, about 27 microns, about 28 microns, about 29 microns, about 30 microns, about 31 microns, about 32 microns, about 33 microns, about 34 microns, about 35 microns, about 36 microns, about 37 microns, about 38 microns, about 39 microns, about 40 microns, about 41 microns, about 42 microns, about 43 microns, about 44 microns, about 45 microns, about 46 microns, about 47 microns, about 48 microns, about 49 microns, about 50 microns, about 51 microns, about 52 microns, about 53 microns, about 54 microns, about 55 microns, about 56 microns, about 57 microns, about 58 microns, about 59 microns, or about 60 microns.

The electrically conductive compositions described herein can be made by any suitable method, including a method comprising blending surface oxides formed on a surface of a eutectic gallium alloy into the bulk of the eutectic gallium alloy by shear mixing of the surface oxide/alloy interface. Shear mixing of such compositions can induce a cross linked microstructure in the surface oxides; thereby forming a conducting shear thinning gel composition. A colloidal suspension of micro-structures can be formed within the eutectic gallium alloy/gallium oxide mixture, for example as, gallium oxide particles and/or sheets.

The surface oxides can be blended in any suitable ratio, such as at a ratio of between about 59.9% (by weight) and about 99.9% eutectic gallium alloy, to about 0.1% (by weight) and about 2.0% gallium oxide. For example percentage by weight of gallium alloy blended with gallium oxide is about 60%, 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or greater, such as about 99.9% eutectic gallium alloy while the weight percentage of gallium oxide is about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, and about 2.0% gallium oxide. In embodiments, the eutectic gallium alloy can include gallium-indium or gallium-indium-tin in any ratio of the recited elements. For example, a eutectic gallium alloy can include gallium and indium.

The weight percentage of gallium in the gallium-indium alloy can be between about 40% and about 95%, such as about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, or about 95%.

Alternatively or in addition, the weight percentage of indium in the gallium-indium alloy can be between about 5% and about 60%, such as about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, or about 60%.

A eutectic gallium alloy can include gallium, indium, and tin. The weight percentage of tin in the gallium-indium-tin alloy can be between about 0.001% and about 50%, such as about 0.001%, about 0.005%, about 0.01%, about 0.05%, about 0.1%, about 0.2%, about 0.3%, about 0.4%, about 0.5%, about 0.6%, about 0.7%, about 0.8%, about 0.9%, about 1%, about 1.4%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50%.

The weight percentage of gallium in the gallium-indium-tin alloy can be between about 40% and about 95%, such as about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, or about 95%.

Alternatively or in addition, the weight percentage of indium in the gallium-indium-tin alloy can be between about 5% and about 60%, such as about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, or about 60%.

One or more micro-particles or sub-micron scale particles can be blended with the eutectic gallium alloy and gallium oxide. For example, the one or more micro-particles or sub-micron particles can be blended with the mixture with wt % of between about 0.001% and about 40.0% of micro-particles in the composition, for example about 0.001%, about 0.005%, about 0.01%, about 0.05%, about 0.1%, about 0.2%, about 0.3%, about 0.4%, about 0.5%, about 0.6%, about 0.7%, about 0.8%, about 0.9%, about 1%, about 1.5%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40. In embodiments the particles can be soda glass, silica, borosilicate glass, quartz, oxidized copper, silver coated copper, non-oxidized copper, tungsten, super saturated tin granules, glass, graphite, silver coated copper, such as silver coated copper spheres, and silver coated copper flakes, copper flakes or copper spheres or a combination thereof, or any other material that can be wetted by gallium. In some embodiments the one or more micro-particles or sub-micron scale particles are in the shape of spheroids, rods, tubes, a flakes, plates, cubes, prismatic, pyramidal, cages, and dendrimers. In certain embodiments, the one or more micro-particles or sub-micron scale particles are in the size range of about 0.5 microns to about 60 microns, as about 0.5 microns, about 0.6 microns, about 0.7 microns, about 0.8 microns, about 0.9 microns, about 1 microns, about 1.5 microns, about 2 microns, about 3 microns, about 4 microns, about 5 microns, about 6 microns, about 7 microns, about 8 microns, about 9 microns, about 10 microns, about 11 microns, about 12 microns, about 13 microns, about 14 microns, about 15 microns, about 16 microns, about 17 microns, about 18 microns, about 19 microns, about 20 microns, about 21 microns, about 22 microns, about 23 microns, about 24 microns, about 25 microns, about 26 microns, about 27 microns, about 28 microns, about 29 microns, about 30 microns, about 31 microns, about 32 microns, about 33 microns, about 34 microns, about 35 microns, about 36 microns, about 37 microns, about 38 microns, about 39 microns, about 40 microns, about 41 microns, about 42 microns, about 43 microns, about 44 microns, about 45 microns, about 46 microns, about 47 microns, about 48 microns, about 49 microns, about 50 microns, about 51 microns, about 52 microns, about 53 microns, about 54 microns, about 55 microns, about 56 microns, about 57 microns, about 58 microns, about 59 microns, or about 60 microns.

In other words, as long as the wearable article 104 of the system 100 of FIG. 1 includes features flexible circuits made from the aforementioned deformable conductors, the wearable article 104 can take any form. For example, according to some non-limiting aspects, the wearable article 104 can include a joint monitoring sleeve or brace, such as those described in International Patent Application No. PCT/US2022/071012, titled DEVICES, SYSTEMS, AND METHODS TO MONITOR AND CHARACTERIZE THE MOTIONS OF A USER VIA FLEXIBLE CIRCUITS, and filed Mar. 7, 2022, the disclosure of which is hereby incorporated by reference in its entirety. Alternately, the wearable article 104 of FIG. 1 can include any of the wearable articles described in International Patent Application No. PCT/US2021/071374, titled WEARABLE ARTICLE, filed Sep. 3, 2021 and published on Mar. 10, 2022 as International Patent Publication No. WO2022051776A1. According to still other non-limiting aspects, the wearable article 104 can be configured as a portion or component of a shoe, a sock, a pant, an undergarment, a shirt, a unitard, a sleeve, a jacket, a hat, a wrap, eyeglass, equipment, and/or a patch, amongst any other articles configured to be worn or used by the user 102 within the physical environment 101. The present disclosure further contemplates the wearable article 104 used in conjunction with other wearable articles (not show), such that the system 100 can aggregate signals generated by multiple flexible circuits worn on different body parts of the user 102.

According to the non-limiting aspect of FIG. 1, the physical environment 101 can include a venue in which the user 102 is performing a motion. For example, the physical environment 101 can be a home, a hospital, an office, a field, a court, amongst other locations. In other words, as will be described in further detail herein, the flexible circuits of the wearable article 104 can be configured to generate electrical parameters in association with a motions (e.g., an every-day motion, such as walking or breathing, a prescribed motion, a workplace action, such as typing or an assembly procedure, a an athletic motion, such as running or working out, a prescribed motion, such as an action taken for rehabilitation or medical purposes, and/or a performance-based motion, such as a motion made while playing an instrument or acting, etc.). Because the wearable article 104 is communicably coupled to the computing device 106, the wearable article 104 can transmit signals associated with electrical parameters generated by the wearable article 104 during the performance of the motion to the computing device 106. As previously discussed, the transmission can occur in real-time or retroactively, after the performance. Regardless, the computing device 106 can ultimately receive the signals associated with electrical parameters generated during the motion, which it can subsequently time-stamp. However, according to some non-limiting aspects, the wearable article 104 can time-stamp the data as it is generated and stored in a local memory.

Although the computing device 106 of FIG. 1 is depicted as a server, it shall be appreciated that, according to other non-limiting aspects, the computing device 106 can include a personal computer, a laptop computer, a tablet, a smartphone, and/or a wearable computer, amongst other computing devices. As long as the computing device 106 can be communicably coupled to the wearable article 104 and the one or more data repositories 109, it can use the data generated by wearable article 104 via the flexible circuits. For example, wherein the user 102 wants to decentralize their data via an NFT on a blockchain network 107, the computing device 106 can be used to “mint,” or publish, an NFT on the blockchain network 107 in association with a file that contains the signals (or data) associated with electrical parameters generated during the performance.

It shall be further appreciated that, according to some non-limiting aspects, the system 100 can further include an ancillary device 103 configured for use by the user 102. For example, data generated by the wearable article 104 can be useful to contextualize data generated by the ancillary device 103, which can include a supplemental piece of medical or athletic equipment (e.g., CPAP machine, a heart rate monitor, a blood pressure monitor, a glucose monitor, etc.). The system 100 can, therefore, be configured to not only manage data generated by the wearable article 104, but to merge that data with data generated by the ancillary device 103 to provide unprecedented insights and/or feedback pertaining to the user's 102 use of the wearable article 104, as will be discussed in further detail herein.

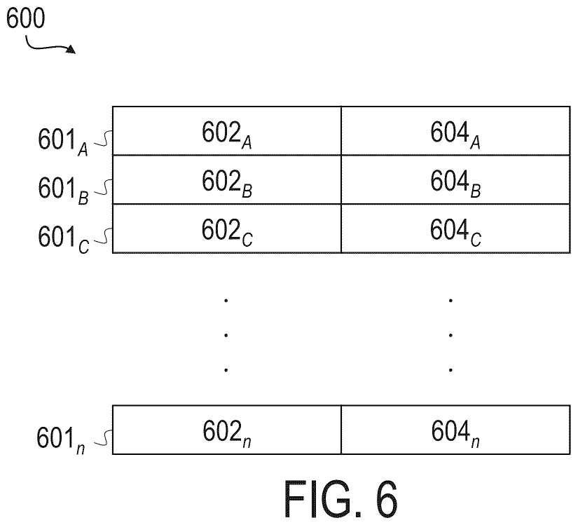

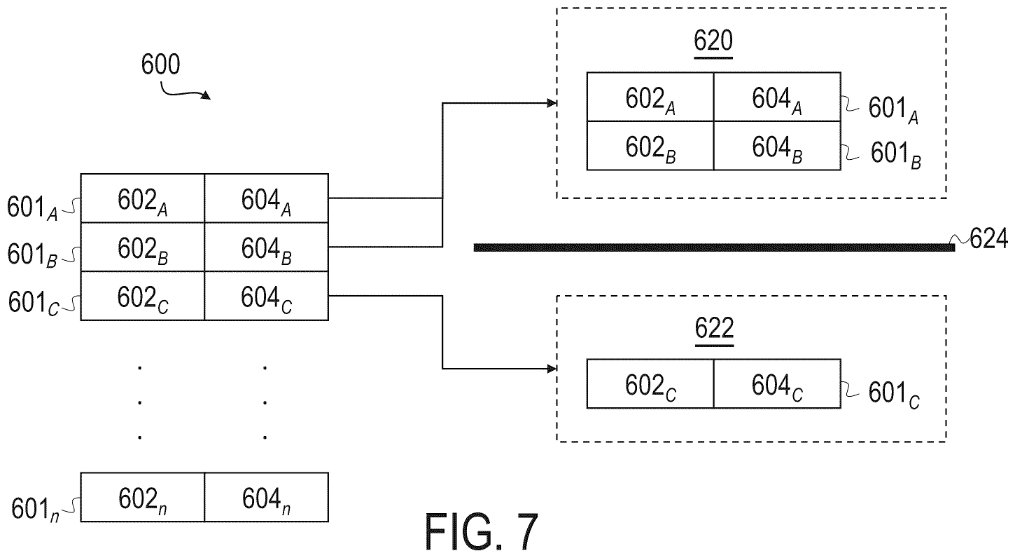

In summary, it shall be appreciated that the system 100 of FIG. 1 is designed to provide the user 102 with unprecedented control over information generated by the wearable article 104. The user 102 is empowered to use a single wearable 104 article for multiple purposes and data generated by the wearable article 104 can be managed by the system 100 for storage in an appropriate repository 107, 111, 113 of the one or more repositories 109 in accordance with one or more predetermined rules, which can be programmed onboard the wearable article 104 or managed via the computing device 106. For example, the rules can include a “triggering event” that, upon recognition by the wearable article 104 or the computing device 106, can result in data being generated by the wearable article 104 to be managed in a particular way, as will be described in further reference to FIGS. 6 and 7. The triggering event can include, for example, a user 102 initiated trigger (e.g., via a physical button or switch on the wearable article 104 or performance of a “wake up” motion recognized by the computing device 106).

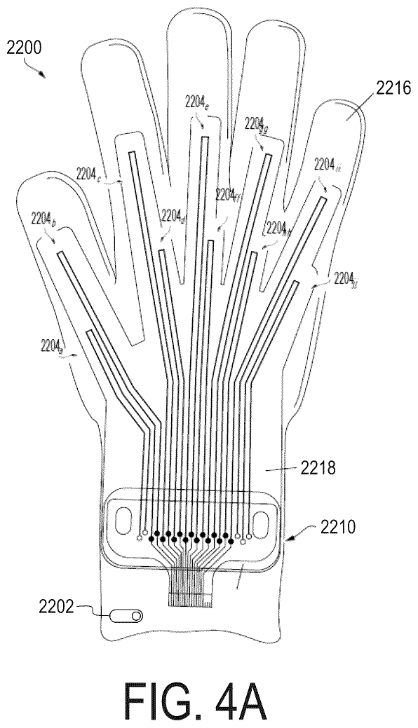

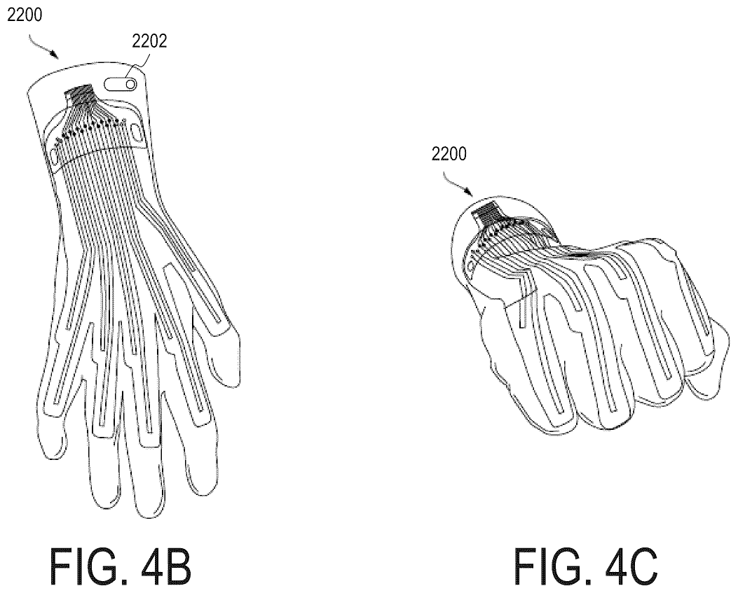

As will be described in further detail with reference to FIGS. 4A-C, the flexible circuits of the wearable article 104 can include traces made from the deformable conductors disclosed here, which generate varying electrical parameters (e.g., an inductance, a resistance, a voltage drop, a capacitance, and an electromagnetic field, etc.). These electrical parameters can be compared and correlated—by the computing device 106, for example—to characterize various physical parameters (e.g., a strain, a stress, a pressure, a dimension, etc.) associated with one or more portions of the wearable article 104 and thus, can characterize the motions of the user's hand. The differences in correlated physical parameters of each circuit can be used, for example, to recognize when the user 102 can performed a triggering motion, which can cause data generated by the wearable article 104 to be distributed to a particular repository 107, 111, 113 of the one or more repositories 109, as described in further reference to FIGS. 6 and 7. As such, based on the varying electrical parameters, the computing device 106 can recognize that the user 102 has either actively (e.g., intentionally performed to initiate the triggering event) or passively (e.g., unconsciously performed, initiating the triggering event regardless of user 102 intention) performed a triggering motion and thus, the computing device 106 can disposition and/or manage data generated by the wearable article 104 according to the predetermined rules.

For example, upon initiation of the triggering event, the computing device 106 of the system 100 of FIG. 1 can be configured to disposition data generated by the wearable article 104 to either the personal server 111, the blockchain network 107, or the third-party server 113, in accordance with the predetermined rules. The predetermined rules could include a first triggering event, for example, indicating that the user 102 is performing a routine physical exercise (e.g., running, lifting weights, yoga, etc.) for their own personal and/or recreational purposes. Accordingly, the computing device 106 can ensure that data generated by the wearable article 104, while the user 102 is performing the routine physical exercise, is dispositioned to the personal server 111. However, the predetermined rules could include a second triggering event, for example, indicating that the user 102 is performing a medical motion (e.g., a physical therapy exercise, a therapeutic exercise, a medically concerning motion, such as breathing at an elevated rate, etc.) that should be monitored by a doctor of the user 102 for medical purposes. Accordingly, the computing device 106 can ensure that data generated by the wearable article 104, while the user 102 is performing the medical motion, is dispositioned to the cryptographically secured blockchain network 107 (e.g., assuming the blockchain network 107 complies with governing laws and regulations) or a third-party server 113, such as a HIPAA compliant server. According to some non-limiting aspects, the predetermined rules can be further configured such that the second triggering event commences collection and/or aggregation of data from the ancillary device 103, as well, which can supplement and thus, enhance, insights gleaned from data generated by the wearable article 104. In still other non-limiting aspects, the triggering event can even include a detection, performed by the computing device 106, of the ancillary device 103 being activated.

According to other non-limiting aspects, the predetermined rules could include a third triggering event, for example, indicating that the user 102 is performing a motion of interest to a third-party, such as a heath insurance company of the user 102. The motion, for example, can include any exercise or motion deemed relevant to an insurance policy of the user 102. Accordingly, the computing device 106 can ensure that data generated by the wearable article 104, while the user 102 is performing the motion of interest, is dispositioned to the third-party server 113, such as a server of the insurance company. It shall be appreciated that these non-limiting aspects are merely illustrative and the predetermined rules and one or more repositories 109 can be alternately configured to comply with any requirements of any entity, for example, such as the Family Educational Rights and Privacy Act (“FERPA”), or workman's compensation requirements imposed by an employer. In fact, the system 100 of FIG. 1 can accommodate any number of rules to ensure compliance with any number of requirements imposed by any number of entities. For example, the triggering event can include a detection, by the computing device 106, that a workman's compensation claim has been generated, or potentially stored one the third-party server 113.

Accordingly, the system 100 can enhance the number of applications in which the wearable article 104 can be implemented and remain in compliance with user 102 requirements, requirements imposed by a third-party server 113, and/or laws or regulations. Such requirements, laws, and/or regulations can be embodied in the one or more rules provided via user 102 input to the computing device 106 for management of data generated by the wearable article 104. Moreover, the system 100 can enable the user 102, or any other user of the system 100 (e.g., a doctor, an insurer, an employer, a government, etc.) to contextualize data generated by the wearable article 104 with data generated by the ancillary device 103. Instead of necessitating a different wearable article for ever application governed by a different set of rules, the same wearable article 104 and system 100 can be used, reducing waste and enhancing the user 102 experience.

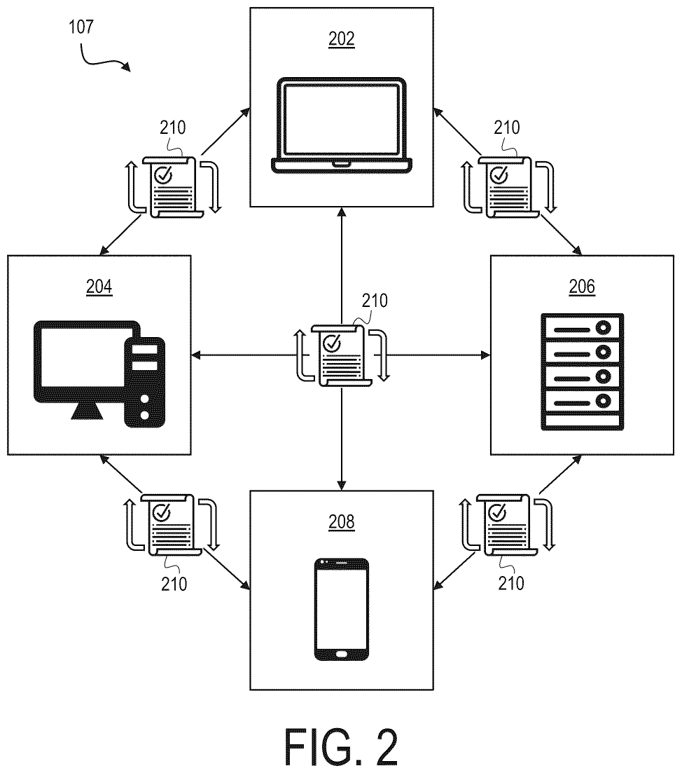

Referring now to FIG. 2, a block diagram of a system for implementing a blockchain network 107 configured to host an NFT is depicted in accordance with at least one non-limiting aspect of the present disclosure. According to the non-limiting aspect of FIG. 2, the blockchain network 107 can include one or more nodes 202, 204, 206, 208 configured to interact with each other such that the nodes 202, 204, 206, 208 can collectively host, modify, and verify a distributed ledger 210. For example, according to the non-limiting aspect of FIG. 2, the blockchain network 202 can include one or more laptop computers 202, personal computers 204, servers 206, and/or mobile computing devices 208, such as a smart phone and/or a tablet. However, it shall be appreciated that the non-limiting aspect of FIG. 2 is merely illustrative. As such, the blockchain network 107 can include any number and/or type of nodes 202, 204, 206, 208 necessary to effectively host, modify, and verify a distributed ledger 210. Moreover, certain privileges associated with the distributed ledger 210 can be selectively allocated to certain nodes 202, 204, 206, 208 of the blockchain network 107. For example, most notes may be configured only to verify or validate the distributed ledger 210, while a select number of nodes may have the ability to modify the distributed ledger 210 and/or generate new blocks.

According to the non-limiting aspect of FIG. 2, the distributed ledger 210 can include records of files (e.g., files containing data generated by the wearable article 104 of FIG. 1) and/or transactions conducted between accounts associated with the blockchain network 107. For example, the distributed ledger 210 can include records associated with transactions executed via smart contracts, or code that automatically executes all components of an agreement that is then stored in the distributed ledger 210. According to some non-limiting aspects, the computing device 106 (FIG. 1) can generate an NFT associated with data generated by the wearable article 104 and the aforementioned rules can be programmed into a smart contract that governs how the NFT can be accessed, managed, or otherwise transacted. The code itself can be replicated across the multiple nodes 202, 204, 206, 208 of a blockchain network 107 and, therefore, the distributed ledger 210 and its records benefit from the security, permanence, and immutability provided by the blockchain 107. AN NFT can be the subject of transactions hosted by the distributed ledger 210. Notably, the blockchain network 107 can include any foundational, “layer two,” or tributary chain, including chains such as the Bitcoin blockchain, Ethereum, Polygon, Arbitrum, and/or Loopring, amongst others.

In further reference to FIG. 2, a user operating a user device (e.g., one of the nodes 202, 204, 206, 208) or a computing device in communication with a node 202, 204, 206, 208, can initiate a transaction by generating a cryptographically signed message and sending the message to the blockchain network 107. The message can include transaction data such as information pertaining to an object of the transaction (e.g., a cryptocurrency, an NFT, etc.), a recipient, and/or an amount associated with the transaction, amongst other information. Once a node 202, 204, 206, 208 receives the message, the node 202, 204, 206, 208 can distribute the message to the other nodes 202, 204, 206, 208 in the blockchain network 107.

According to some non-limiting aspects, each of the nodes 202, 204, 206, 208 of the blockchain network 107 can include the transaction represented in the generated message in a block of other transactions and can attempt to validate or cryptographically solve the block. The first node 202, 204, 206, 208 that solves the block can provide the solution to the other validation nodes for verification, and ledger 210 maintained at each of the nodes 202, 204, 206, 208 can be updated to add the block to the distributed ledger 210 to effect the transaction. As an incentive to cryptographically solve blocks—which consumes electricity and computing resources—select nodes 202, 204, 206, 208 can earn at least a part of a token hosted on the distributed ledger 210 (e.g., a cryptocurrency) and/or a fee for participating in the validation of the block.

As such, it shall be appreciated that the distributed ledger 210—and more generally, the blockchain network 107—of FIG. 2 can be used to track transactions and ownership of any number of digital assets, including NFTs and other files generated by the computing device 106 (FIG. 1) that include data generated by the wearable article 104 (FIG. 1). In other words, because the computing device 106 of FIG. 1 is configured to interface with the blockchain network 107 of FIG. 2, the computing device 106 can create an NFT on the blockchain network 107 in association with a file that contains the signals (or data) associated with electrical parameters generated during the performance. Moreover, via the blockchain network 107 of FIG. 2, exclusive ownership of the NFT can be tracked with enhanced security provided by the distributed ledger 210. Additionally, via smart contracts, the blockchain network 107 can be used by (or in conjunction with) the computing device 106 (FIG. 1) to enforce one or more predetermined rules, thereby ensuring compliance with governing requirements, rules, and/or regulations. In other words, via the blockchain network 107, accesses and privileges to data generated by the wearable article 104 can be managed. For example, according to one non-limiting aspect, the blockchain network 107, itself, can be configured to be compliant with a governing regulation (e.g., HIPAA, FERPA, etc.) and thus, used in lieu of a traditionally secured and otherwise compliant third-party server 113 (FIG. 1), such as an electronic medical record (“EMR”) server.

Each NFT can include a public key and/or a private key, amongst other cryptographic information that can be used to identify and verify ownership of an NFT hosted on the blockchain network 107. The system 100 of FIG. 1 can use the public key cryptography to locate the NFT on the blockchain network 107. However, every public key matches to only one private key and thus, exclusive ownership of the NFT—and thus, the file that contains the signals (or data) associated with electrical parameters generated during the performance—can be only confirmed via the private key. Moreover, the NFT cannot be accessed or transacted without the private key, further enhancing the security of the file containing signals associated with electrical parameters generated during the performance. The NFT can be useful, for example, because the signals (or data) associated with electrical parameters generated during the performance can be used to simulate the user's 104 (FIG. 1) motions during the performance in the physical environment 101 (FIG. 1) in a virtual environment. According to other non-limiting aspects, the term “exclusive ownership” can include a registered or authenticated ownership of an NFT asset via the blockchain network 107. For example, a single NFT can be one of a plurality of NFTs issued in association with the same multimedia file. In still other non-limiting aspects, the NFTs of a plurality of NFTs can be serialized, indicating a limited number of assets available. It shall be appreciated that some consumers may value lower serial numbers from the plurality of NFTs over higher serial numbers. Additionally and/or alternatively, an NFT can be used to identify a specific wearable article, for example, by including a serial number of the wearable article and/or a digital certificate of ownership associated with a serial number of the wearable article. Accordingly, the NFT can authenticate the ownership of a particular wearable article, which can be useful wherein wearable articles are sold as collectibles or implemented for medical treatment of a particular patient, for example.

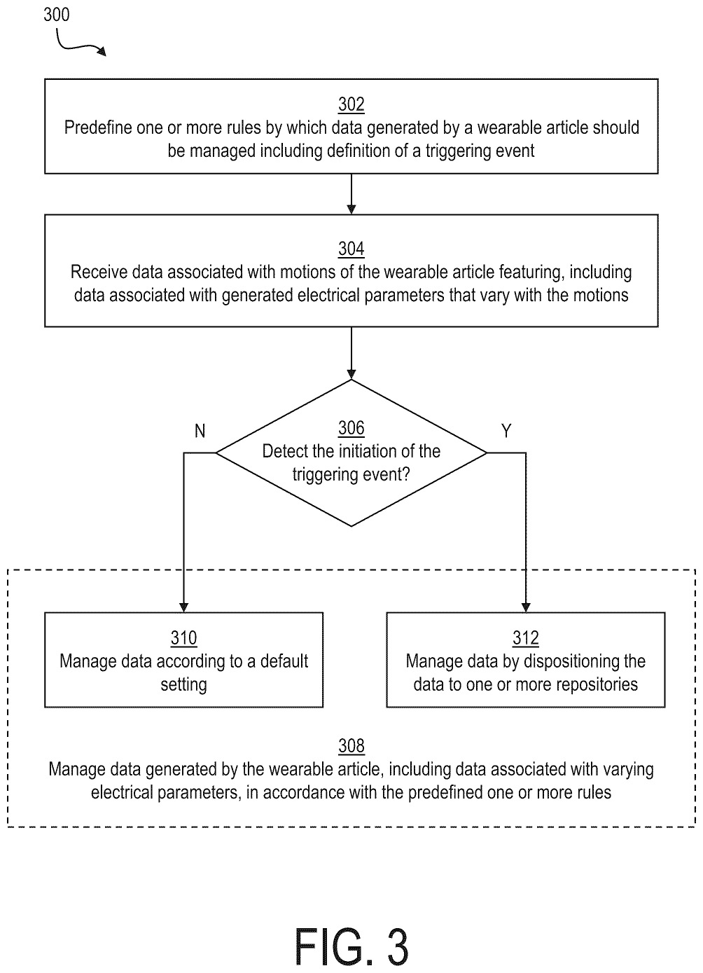

Referring now to FIG. 3, a method 300 of autonomously dispositioning data generated by a single wearable article in compliance with multiple application-specific requirements is depicted in accordance with at least on non-limiting aspect of the present disclosure. According to some non-limiting aspects, the method 300, for example, can be performed by the computing device 106 of the system 100 of FIG. 1. However, according to other non-limiting aspects, the method 300 can be performed by a processor communicably coupled to the wearable article 104 of the system 100 of FIG. 1. Regardless, the method 300 can include predefining 302 one or more rules by which data generated by a wearable article should be managed including definition of a triggering event. Such rules can be defined via one or more user inputs provided via the computing device 106 (FIG. 1) or a user interface of the wearable article 104 (FIG. 1), itself. Alternately, predefining 302 the one or more rules can include programming a smart contract governed by the blockchain network 107 (FIG. 1).

Still referring to FIG. 3, the method 300 can further include receiving 304 data associated with motions of the wearable article featuring, including data associated with generated electrical parameters that vary with the motions. As previously described, the wearable article can include flexible circuits with traces formed of the deformable conductors disclosed herein, which generate varying electrical parameters (e.g., an inductance, a resistance, a voltage drop, a capacitance, and an electromagnetic field, etc.) as they move. These electrical parameters can be compared and correlated—by the computing device 106 (FIG. 1), for example—to characterize various physical parameters (e.g., a strain, a stress, a pressure, a dimension, etc.) associated with one or more portions of the wearable article 104 and thus, can characterize the motions of the user 102 (FIG. 1). The differences in correlated physical parameters of each circuit can be used, for example, to recognize when the user 102 can performed a triggering motion, which can cause data generated by the wearable article 104 to be distributed to a particular repository 107, 111, 113 of the one or more repositories 109, as described in further reference to FIGS. 6 and 7.

In further reference to FIG. 3, the method 300 can include detecting 306 an initiation of the triggering event. As previously described, the triggering event can, for example, include an engagement with a physical button and/or switch on the wearable article 104 (FIG. 1), a user 102 (FIG. 1) either actively or passively performing a triggering motion, or detection of another event independent of the wearable article 104 (e.g., activation of an ancillary device 103 (FIG. 1), generation of a claim via the third-party server 113 (FIG. 1), etc.). The method 300 can further include managing 308 data generated by the wearable article, including data associated with varying electrical parameters, in accordance with the predefined one or more rules.

According to the non-limiting aspect of FIG. 3, assuming the triggering event is detected, managing 308 the data generated by the wearable article in accordance with the predefined one or more rules the method 300 can include dispositioning 312 the data to one or more predefined repositories. For example, wherein the triggering event indicates that the user 102 (FIG. 1) is performing a routine physical exercise (e.g., running, lifting weights, yoga, etc.) for their own personal and/or recreational purposes, data generated by the wearable article 104 (FIG. 1) can be dispositioned to the personal server 111 (FIG. 1). However, wherein the triggering event indicates that the user 102 is performing a medical motion (e.g., a physical therapy exercise, a therapeutic exercise, a medically concerning motion, such as breathing at an elevated rate, etc.) that should be monitored by a doctor of the user 102 (FIG. 1) for medical purposes data generated by the wearable article 104 can be dispositioned to the cryptographically secured blockchain network 107 (e.g., assuming the blockchain network 107 complies with governing laws and regulations) or a third-party server 113, such as a HIPAA compliant server. Wherein the triggering event indicates that the user 102 is performing a motion of interest to a heath insurance company of the user 102 (FIG. 1), data generated by the wearable article 104 (FIG. 1) can be dispositioned to the third-party server 113 (FIG. 1), such as a server of the insurance company. Alternately, the triggering event can include engagement by the user 102 (FIG. 1) with a button 2202 (FIG. 4A) and thus, data generated by the wearable article 104 (FIG. 1) can be dispositioned to any of the one or more repositories 109 (FIG. 1). Once again, these examples are merely illustrative and any combination of rules and/or triggering events imposed by any entity can be implemented via the method 300 of FIG. 3.

However, in the absence of a detection that the triggering event as been initiated, the method 300 can further include managing 308 data generated by the wearable article 104 (FIG. 1) in accordance with a default setting of the wearable article 100 (FIG. 1). For example, absent detection of the triggering event, the system 100 (FIG. 1) can be configured to disposition data generated by the wearable article 104 (FIG. 1) to the personal server 111 (FIG. 1), by default. Of course, such default settings can be altered in accordance with user preference and/or intended application. The default setting may, for example, be a disposition of data generated by the wearable article 104 (FIG. 1) to the blockchain network 107 (FIG. 1) by minting the data into one or more NFTs, such that predetermined rules programmed into a smart contract can further govern how the data is accessed and/or otherwise managed.

Referring now to FIGS. 4A-C, a wearable article 2200 configured for use via the system 100 of FIG. 1 is depicted in accordance with at least one non-limiting aspect of the present disclosure. According to the non-limiting aspect of FIGS. 4A-C, the wearable article 2200 can be configured as a glove that features flexible circuits 2204ₐ₋ⱼ and can be configured to be worn on a user's hand. The glove 2200 can include flexible circuits 2204ₐ₋ⱼ that utilize deformable conductors to generate electrical parameters, which can be correlated to physical parameters associated with a user's physical movements when wearing the glove. Of course, according to other non-limiting aspects, the article can take the form of any other article of clothing, including a knee brace, a shirt, pants, a sock, and/or a hat, amongst others.

In further reference to FIGS. 4A-C, the glove 2200 can include a plurality of circuits 2204ₐ₋ₑ including a network of traces that are specifically configured to traverse various geometrical portions of the glove 2200. The glove 2200 of FIGS. 4A-C can include ten circuits 2204ₐ₋ⱼ, each with a network of elongated, looping traces mounted to a substrate 2018. According to some non-limiting aspects, the circuits 2204ₐ₋ₑ, including the traces and substrates 2218, can be constructed as described U.S. patent application Ser. No. 16/548,379 titled STRUCTURES WITH DEFORMABLE CONDUCTORS, which was filed on Aug. 22, 2019 and granted as U.S. Pat. No. 11,088,063 on Aug. 10, 2021, the disclosure of which is hereby incorporated by reference in its entirety. The traces of FIGS. 4A-C can include any deformable conductor, such as those disclosed in International Patent Application No. PCT/US2017/019762 titled LIQUID WIRE, which was filed on Feb. 27, 2017 and published on Sep. 8, 2017 as International Patent Publication No. WO2017/151523A1, the disclosure of which is hereby incorporated by reference in its entirety.

Notably, the traces of FIGS. 4A-C can be particularly configured such that, while wearing the glove 2200, a user's motions can result in deformation of the elongated traces which can alter electrical parameters that can be correlated to baseline data. Each circuit 2204ₐ₋ₕ has a trace with a desired length. For example, the trace of the first circuit 2204ₐ, fourth circuit 2204ₐ, sixth circuit 2204_{f}, eighth circuit 2204ₕ, and tenth circuit 2204ⱼ are comparatively shorter than the second circuit 2204_{b}, third circuit 2204_{c}, fifth circuit 2204ₑ, seventh circuit 2204_{g}, and ninth circuit 2204ᵢ. The trace of the first circuit 2204ₐ, fourth circuit 2204ₐ, sixth circuit 2204_{f}, eighth circuit 2204ₕ, and tenth circuit 2204ⱼ extend to a first location of interest, approximately, where a user's most proximal knuckle of each finger would be positioned. Likewise, the second circuit 2204_{b}, third circuit 2204_{c}, fifth circuit 2204ₑ, seventh circuit 2204_{g}, and ninth circuit 2204ᵢ extend to a second location of interest, approximately, where a user's intermediate knuckle of each finger would be positioned. Accordingly, electrical parameters (e.g., an inductance, a resistance, a voltage drop, a capacitance, and an electromagnetic field, etc.) generated by the traces of each circuit 2204ₐ₋ⱼ can be compared and correlated to physical parameters (e.g., a strain, a stress, a pressure, a dimension, etc.) associated with one or more portions of the glove 2200 and thus, can characterize the motion of the user's hand. The differences in correlated physical parameters of each circuit 2204ₐ₋ⱼ can be used to model the user's hand in a virtual environment.

Additionally, as depicted in FIG. 4A, according to some non-limiting aspects, the glove 2200 can include a physical switch 2202 (or a button or any other electrical means or mechanism) that, upon activation of the user 102 (FIG. 1), can initiate a “triggering event.” As previously described, upon recognition of the triggering event, data generated by the glove 2200 can be handled in a particular way according to the predetermined rules, as will be described in further reference to FIGS. 6 and 7. However, according to other non-limiting aspects, the physical switch 2202 is not physical, but virtually displayed to the user 102 (FIG. 1) via a display communicably coupled to the glove 2200 and/or a computing device 106 (FIG. 1) communicably coupled to the glove 2200. For example, the glove 2200 could be configured to communicate with a computing device 106 (FIG. 1), such as a mobile phone of the user 102 (FIG. 1), via an ad hoc or infrastructure wireless connection and an application executed or otherwise accessed by the mobile phone can present a virtual representation of the physical button 2202.

Although the non-limiting aspects of FIGS. 4A-C depict a glove 2200 that includes circuits 2204ₐ₋ⱼ with varying trace configurations and electrical features, such as a coupling circuit 2210, it shall be appreciated that the present disclosure contemplates other non-limiting aspects, featuring a variety of combinations of the previously disclosed trace configurations and electrical features. For example, the glove 2200 can be alternately configured with a different circuit configuration and a variety of electronic components.

In further reference to FIGS. 4B and 4C, the glove 2200 of FIG. 4A can be used to generate signals including data that corresponds to electrical parameters that vary as the flexible circuits are physically deformed. For example, according to FIG. 4B, the user's hand is relaxed while using the glove 2200. Accordingly, a processor can generate and record signals received from the circuits 2204ₐ₋ⱼ (FIG. 4A) when the glove 2200 is in the first, relaxed position of FIG. 4B. A device capable of generating motion capture data, such as a camera, can be used to record the glove 2200 as the user flexes their hand into a fist, as depicted in FIG. 4C. The processor can subsequently generate and record signals received from the circuits 2204ₐ₋ⱼ (FIG. 4A) when the glove 2200 is in the second, flexed position of FIG. 4C. The processor can correlate the electrical parameter associated with the first, relaxed position of FIG. 4B with the motion capture data associated with the first, relaxed position of FIG. 4B, and the electrical parameter associated with the second, flexed position of FIG. 4C with the motion capture data associated with the second, flexed position of FIG. 4C. Accordingly, the processor can generate a virtual simulation of the user's hand as it transitions from the first, relaxed position of FIG. 4B to the second, flexed position of FIG. 4C, every time the user performs the motion, based on electrical parameters received from the glove 2200 alone, without the assistance of real-time motion capture data generated by a camera. Of course, the use of motion capture data to correlate electrical parameters generated by the glove 2200 of FIGS. 4A-C to the physical motions of the user is only one means of correlating electrical parameters generated by the glove 2200 of FIGS. 4A-C to the physical motions of the user. For example, alternate means can include taking physical measurements of the wearable article 2200, modeling, and utilizing traditional image data, and correlating the resulting data to electrical parameters generated by the glove 2200 of FIGS. 4A-C to the physical motions of the user.

It shall be appreciated that wearable articles, such as the glove 2200 of FIG. 4A-C can be used to simulate the motions of user in a virtual environment. This can provide numerous benefits due to a reduction of ancillary components required to simulate the user's motions while in use. For example, conventional articles may rely on a plurality of IMUs, gyroscopes, and/or accelerometers to estimate the articles position and/or orientation in space. However, such components can be bulky and/or uncomfortable for the user and may have increased requirements causing the article to be impractical and inefficient for everyday use. As such, there is a need for devices, systems, and methods for simulating motions in a virtual environment using a wearable article with flexible circuits. The flexible circuits can reduce the number of ancillary components needed to simulate the user's motions in a virtual environment and thus, can result in a more streamlined fit that requires less power to achieve the same, or enhanced results.

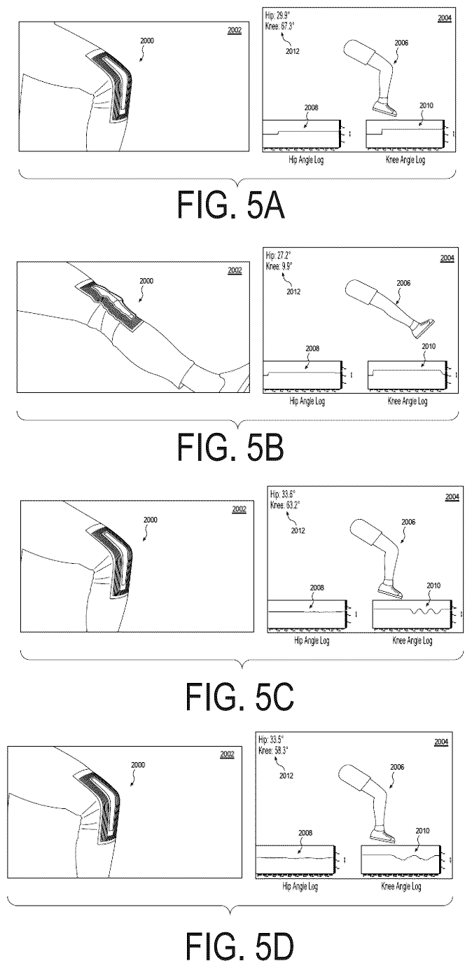

Referring now to FIGS. 5A-D, another wearable article 2000 configured for use with the system 100 of FIG. 1, including a corresponding characterization 2006 of the monitored motions, is depicted in accordance with at least one non-limiting aspect of the present disclosure. Specifically, FIGS. 5A-D depict how NFTs minted by the blockchain network 107 of FIGS. 1 and 2, including the data associated with the various electrical parameters generated by the wearable article 104 (FIG. 1) during a performance, can be used to simulate the user's 102 (FIG. 1) motions during the performance via the method 300 of FIG. 3. For example, a wearable article 2000 configured as a joint monitoring sleeve is depicted in an actual environment 2002. According to the non-limiting aspect of FIGS. 5A-D, the joint monitoring sleeve 2000 can include a flexible circuit 2001 configured as a strain sensor dispositioned across a user's knee. However, according to other non-limiting aspects, the joint monitoring sleeve 2000 can further include any number of electrodes, IMUs, pressure sensors, and/or temperature sensors, as described herein.

Additionally, FIGS. 5A-D further depict a generated model 2006 of the joint monitoring sleeve 2000 in a virtual environment 2004. As previously described, the flexible circuit 2001 can generate electrical parameters and it is deformed while the user is moving their leg, and the electrical parameters can be used to generate a highly accurate model 2006 of the joint monitoring sleeve 2000 based on correlations, as described in the method 300 of FIG. 3. The model 2006 can be presented on a display communicably coupled to a processor, along with various widgets 2008, 2010, 2012. For example, a first widget 2012 can present real-time motion data associated with the current condition of the user's joint and/or appendage. For example, according to the non-limiting aspect of FIG. 5A, the user's leg is bent within the joint monitoring sleeve 2000. Accordingly, the first widget 2012 displays a current hip angle of 29.9 degrees and a current knee angle of 67.3 degrees. The second widget 2008 and the third widget 2010 are historical motion data charts and thus, exclusively reflect the current hip angle and knee angle since the monitoring and characterization has just begun. Additionally, the generated model 2006 of the user's leg reflects the real-time position of the user's leg with a hip angle of 29.9 degrees and a knee angle of 67.3 degrees, within the joint monitoring sleeve 2000.

Referring now to FIG. 5B, the user has extended their leg within the joint monitoring sleeve 2000 in the actual environment. Accordingly, the first widget 2012 indicates that the user's current hip angle is 27.2 degrees and current knee angle is 9.9 degrees, and the model 2006 has been updated to accurately reflect the real-time position of the user's leg within the joint monitoring sleeve 2000 in the virtual environment 2004. Moreover, the second widget 2008 and third widget 2010 have been updated to reflect the change in the historical motion data monitored and characterized by the joint monitoring sleeve 2000. In FIG. 5C, the user has once again bent their knee to a hip angle of 33.6 degrees and a knee angle of 63.2 degrees. In the virtual environment 2004, the model 2006 and first widget 2012 have been updated accordingly to reflect the real-time position of the user's leg within the joint monitoring sleeve 2000. Additionally, the second widget 2008 and third widget 2010 have been updated to log the real-time position data on the historical chart.

According to FIG. 5D, the user has continued the hip flexions of FIGS. 5A-C a few times, as is illustrated via the second widget 2008 and third widget 2010. Aside from the generated model 2006 characterizing the real-time position of the user's leg within the joint monitoring sleeve 2000 in the actual environment, the second widget 2008 and third widget 2010 have been updated to reflect a sinusoidal-type curve of significantly high resolution, which illustrates the accuracy with which the user's motion within the joint monitoring sleeve 2000 can be monitored. As such, it shall be appreciated how the integration of various combinations of flexible circuits, sensors, and/or electronic components into a wearable article, as disclosed herein, can be implemented to generate highly accurate models of a user's motions. This can produce numerous benefits. For example, according to some non-limiting aspects, a doctor can monitor a patient's rehabilitation from a remote location, increasing access to high-quality health care. According to other non-limiting aspects, the model 2006 of FIGS. 5A-D can be used for virtual reality games and/or other applications, including improved metaverse applications. According to some non-limiting aspects, the model 2006 and/or widgets 2008, 2010, 2012 can be displayed on a mobile computing device. As previously discussed, the virtual environment 2004 can be a graphical user interface, such as an electronic trading card or any virtual environment and/or a virtually augmented physical environment, such as the metaverse or a video game. Accordingly, the consumer of the NFT can be the exclusive owner of the model depicted in FIGS. 5A-D.

As previously described, according to some non-limiting aspects, it might be beneficial to store data generated by a wearable article featuring flexible circuits in an NFT for health-related applications. For example, as previously discussed, data can be generated by a wearable article featuring flexible circuits and thus, that data can be associated with electrical the motions of a user while wearing the wearable article. However, according to some non-limiting aspects, the user may not be a performer but an average healthcare consumer. The average healthcare consumer may utilize wearable articles with flexible circuits in a variety of different situations.

For example, the average healthcare consumer might utilize a wearable article with flexible circuits for athletic purposes, to monitor, track, and characterize their performance in ways similar to those previously discussed. The average healthcare consumer, however, may also utilize a wearable article with flexible circuits for medical and/or rehabilitative purposes. Thus, it is important that data generated by the wearable article with flexible circuits can be marked, indexed, and segmented for confidential purposes and/or applications, in compliance with laws and regulations, such as the Health Insurance Portability and Accountability Act of 1996 (“HIPAA”). Accordingly, there is a need for devices, systems, and methods for creating and managing health records using NFTs and data generated by flexible circuits.

Referring now to FIG. 6, a table 600 of data generated by a wearable article featuring flexible circuits is depicted in accordance with at least one non-limiting aspect of the present disclosure. For example, the wearable article can include any of the wearable articles discussed herein, such as the wearable article 2200 of FIGS. 4A-C or the wearable article 2000 of FIGS. 5A-D. Likewise, the flexible circuits can include traces made from any of the deformable conductors discussed herein. According to the non-limiting aspect of FIG. 6, the table 600 can include a plurality of data entries 601_{A-n}, wherein each data entry 601_{A-n} corresponds to electrical parameters (e.g., an inductance, a resistance, a voltage drop, a capacitance, and an electromagnetic field, etc.) generated by the traces of each flexible circuit while the user is moving while wearing the wearable article.

Additionally, according to the non-limiting aspect of FIG. 6, data entry 601_{A-n} can include a number of components 602_{A-n}, 604_{A-n} configured to assist in indexing the data 601_{A-n} for different purposes according to different applications. Each data entry 601_{A-n} can include, at a minimum, a key component 602_{A-n} and a value component 604_{A-n}. The value component 604_{A-n}. can correspond to the specific electrical parameter (e.g., an inductance, a resistance, a voltage drop, a capacitance, and an electromagnetic field, etc.) generated by the traces of each flexible circuit while the user is moving while wearing the wearable article. However, the key component 602_{A-n} can include a searchable variable that distinguishes each data entry 601_{A-n}—and more specifically, the value component 604_{A-n} of each data entry 601_{A-n}—from others in the table 600. In other words, the key component 602_{A-n} can include a searchable tag that provides some degree of context for the value component 604_{A-n} of each data entry value component 601_{A-n}. For example, the key component 602_{A-n} can include a time-stamp, a geo-location stamp, a device-identifying stamp, and/or any other metadata configured to provide context as to how, where, and when the electrical parameter associated with the value component 604_{A-n} was generated.

Still referring to FIG. 6, the key component 602_{A-n} can be either generated by the wearable article, autonomously generated by a local or cloud computer, or manually assigned to each value component 604_{A-n}. For example, according to some non-limiting aspects, the wearable article can include one or more electronic components—such as a microprocessor, a geo-location device, and/or a clock—configured to generate the key component 602_{A-n} (e.g., time-stamp, geo-location stamp, device-identifying stamp, etc.) and associate with each value component 604_{A-n} of each data entry 601_{A-n} as generated by the flexible circuits. Alternately and/or additionally, the value component 604_{A-n} of each data entry 601_{A-n} can be autonomously annotated with a key component 602_{A-n} by an ancillary computing device communicably coupled to the wearable article. For example, the computing device can be either local or remotely located relative to the wearable article. According to still other non-limiting aspects, each value component 604_{A-n} can be manually annotated by a user of the wearable article or a third-party via a computing device. Regardless, each value component 604_{A-n} of each data entry 601_{A-n} can be accompanied by a searchable key component 602_{A-n}. As such, the data entries 601_{A-n} of FIG. 6—including their respective key components 602_{A-n} and value components 604_{A-n}—can be minted into an NFT hosted on a distributed ledger by a blockchain network, as previously described in reference to FIG. 2.

According to still other non-limiting aspects, each data entry 601_{A-n} can be accompanied by a patient-identifying component. The key component 602_{A-n} can include a patient-identifying component that accompanies the value component 604_{A-n}, such that each value component 604_{A-n} is attributable to a particular user of the wearable article during the generation of the value component 604_{A-n}. For example, according to such aspects, a wearable article can include a biometric sensor (e.g., a fingerprint scanner, a heart-rate sensor, sensors configured to detect images and/or sounds associated with the user, etc.). According to some non-limiting aspects, a computing device communicably coupled to the wearable article can identify the user's voice and/or face based on sounds and/or images detected by such sensors. As such, biometric data generated based on signals received from such biometric sensors can be included in the key component 602_{A-n}, such that the key component 602_{A-n} is user identifying. It shall be appreciated that including such user identifying data in the key component 602_{A-n} of each data entry 601_{A-n} can be particularly beneficial in non-limiting aspects where the wearable article is used for medical purposes, as the key component 602_{A-n} can be used to authenticate the user of a wearable article, or patient, as it may be. In other words, user identifying data in the key component 602_{A-n} of each data entry 601_{A-n} can intrinsically link the patient—who wore the wearable article—to motion-based data in the value component 604_{A-n}. This can greatly increase confidence in each data entry 601_{A-n} and can significantly reduce the possibility of fraud.

According to other non-limiting aspects, the value component 604_{A-n} of each data entry 601_{A-n} itself, can be user identifying. For example, value component 604_{A-n} can include data based on signals generated by the flexible circuits of a wearable article and thus, can be correlated to the physical activity of a user of the wearable article. An average and/or mean value associated with the value components 604_{A-n} of each data entry 601_{A-n} can be generated and used to characterize the physical abilities of the user. Additionally, the key component 602_{A-n} of each data entry 601_{A-n} can include a time stamp that indicates when the value components 604_{A-n} were generated by the wearable article. As such, if the a value component 604_{A-n} of a data entry 601_{A-n} deviates significantly from the calculated mean and/or average value components 604_{A-n} for a particular user, and the key component 602_{A-n} of that data entry 601_{A-n} indicates that the deviating value component 604_{A-n} was generated within a predetermined temporal proximity relative to the value components 604_{A-n} used to generate the mean and/or average, the magnitude of the deviation may be indicative of a different user having used the wearable article to generate the deviating value component 604_{A-n}.

For example, the calculated mean and/or average value components 604_{A-n} may indicate that a user has a particular physical capability or feature (e.g., range of flexibility, or a particular heart rate, lung volume, waistline, etc.). If the value component 604_{A-n} of a suspect data entry 601_{A-n} is indicative of a significant deviation from that physical capability or feature, and the key component 602_{A-n} (e.g., time-stamp, etc.) of the suspect data entry 601_{A-n} indicates that the deviation occurred too soon, a third party (e.g., doctor, auditor, referee, insurer, etc.) may conclude that a different user was wearing the wearable article when the suspect data entry 601_{A-n} was generated. In other words, based on the key component 602_{A-n} and the value component 604_{A-n} of the suspect data entry 601_{A-n}, a third party may determine that the user progressed too fast too soon, for example, and reasonably conclude that the user could not be responsible for the suspect data entry 601_{A-n}.

Similarly, it shall be appreciated that a relative comparison of the sizes of the key component 602_{A-n} and the value component 604_{A-n} of a particular data entry 601_{A-n} can be indicative of a user's activity while wearing the wearable article. For example, if the value component 604_{A-n} of the data entry 601_{A-n} includes a large quantity of data, it can indicate that the user was very active. Likewise, if the key component 602_{A-n} of the data entry 601_{A-n} includes a small quantity of data, it can indicate that the user only used the wearable article for a short period of time. Accordingly, if the value component 604_{A-n} of the data entry 601_{A-n} includes a large quantity of data and the key component 602_{A-n} of the data entry 601_{A-n} includes a small quantity of data, it can indicate that the user was extremely active over a short period of time. For example, a third-party may reasonably conclude, based on the data entry 601_{A-n}, that the user was playing a soccer game, or went for a run. Conversely, if the value component 604_{A-n} of the data entry 601_{A-n} includes a small quantity of data and the key component 602_{A-n} of the data entry 601_{A-n} includes a large quantity of data, it can indicate that the user was sedentary over a longer period of time. In other words, the size of the key component 602_{A-n} relative to the size of the value component 604_{A-n} can be used to characterize the activity associated with a particular data entry 601 _{A-n}.

It shall be appreciated that, since each NFT can include a public key and/or a private key, amongst other cryptographic information that can be used to identify and secure the content of the NFT, the blockchain network 107 (FIG. 2) can provide a beneficial means of storing information that needs to be properly indexed and—depending on the particular application—secured for confidentiality. For example, along with the searchable key components 602_{A-n} a public key can be associated with NFTs containing one or more of the data entries 601_{A-n} of FIG. 6 to facilitate the location of each NFT on the blockchain network 107, while cryptographically securing the content of each NFT via the private key. Since each public key matches to only one private key, access to the content of each NFT—and thus, the file that contains the value components 604_{A-n}—can be only attained via the private key. Moreover, the NFT cannot be accessed or transacted without the private key, further enhancing the security of the file containing the value components 604_{A-n}. As such, the blockchain network 107 (FIG. 2) can be used to store information generated by a wearable article while securing it for confidentiality, as will be described in further detail with reference to FIG. 7.

Referring now to FIG. 7, a means of indexing the data entries 601 _{A-n} of the table 600 of FIG. 6 vian NFTs is depicted in accordance with at least one non-limiting aspect of the present disclosure. According to the non-limiting aspect of FIG. 7, the data entries 601_{A-n} of the table 600 of FIG. 6 can be segregated based on the searchable key components 602_{A-n} such that the value components 604_{A}, 604_{B} of certain data entries 601_{A}, 601_{B} are placed in a confidential storage 620 such that they are only accessible to those with a private key associated with a respective NFT. Contrarily, another data entry 601_{C} remains in a non-confidential storage 622 such that its value component 604_{C} remains accessible to those without requiring the private key. According to some non-limiting aspects, the segregation depicted in FIG. 7 can be autonomously performed, such as via the system 100 of FIG. 1 or components thereof. Alternately and/or additionally the segregation depicted in FIG. 7 can be manually performed via a user and/or a third party, such as a doctor and/or insurance provider, amongst others.

In other words, according to the non-limiting aspect of FIG. 7, the data entries 601_{A-n} of the table 600 of FIG. 6 can be sorted and segregated based on the key components 602_{A-n} and treated differently regarding the confidentiality of their respective value components 604_{A-n}. For example, the key components 602_{A-n} (e.g., time-stamps) may indicate that the data entries 601_{A-n} were generated at a particular time. Alternately and/or additionally, the key components 602_{A-n} (e.g., geo-location stamps) may indicate that the data entries 601_{A-n} were generated in a particular location (e.g., a doctor's office or hospital, etc.). Alternately and/or additionally, the key components 602_{A-n} (e.g., device-identifying stamps) may indicate that the data entries 601_{A-n} were generated by a particular wearable article (e.g., a glove or knee-brace, etc.). Any and/or all of these key components 602_{A-n}, amongst others, can be considered when classifying the data entries 601_{A-n} for confidential treatment. For example, the confidential storage 620 of FIG. 7 can include a confidential server on the restricted side of a HIPAA firewall 624. Alternately and/or additionally, the confidential storage 620 of FIG. 7 can include an NFT minted by the blockchain network 107 (FIG. 2) and hosted in a HIPAA compliant way via the distributed ledger 210 (FIG. 2), such that only those with the private key can access the value components 604_{A}, 604_{B} of the confidential data entries 601_{A}, 601_{B}.

It shall be appreciated that selectively segmenting the data entries 601_{A-n} for confidential treatment can be especially beneficial due to the versatility afforded by wearable articles featuring flexible circuits. For example, a user may own a wearable article with flexible circuits for their own personal use, including personal training and/or athletic tracking. Wearable articles such as the knee brace 2000 of FIGS. 5A-D, for example, can be useful to track a various parameters associated with a runner's performance (e.g., form, stride, pace, etc.) However, the knee brace 2000 of FIGS. 5A-D can also provide numerous therapeutic benefits, as data entries 601_{A-n} generated by the knee brace 2000 can be used by third parties (e.g., doctors, physical therapists, insurers, etc.) to monitor a patients recovery and/or other health-related parameters. Thus, the key components 602_{A-n} of the data entries 601_{A-n} generated by a wearable article, such as the knee brace 2000 of FIGS. 5A-D, can be used to identify certain data entries 601_{A}, 601_{B} for exclusive storage in a confidential means 620. For example, according to some non-limiting aspects, the storage of certain data entries 601_{A}, 601_{B} in a confidential means 620 can enable those data entries 601_{A}, 601_{B} to be managed in compliance with a HIPAA firewall 624. However, once the confidential data entries 601_{A}, 601_{B} are offloaded from the wearable article, the user (or patient) is free to continue using the wearable article for personal use.

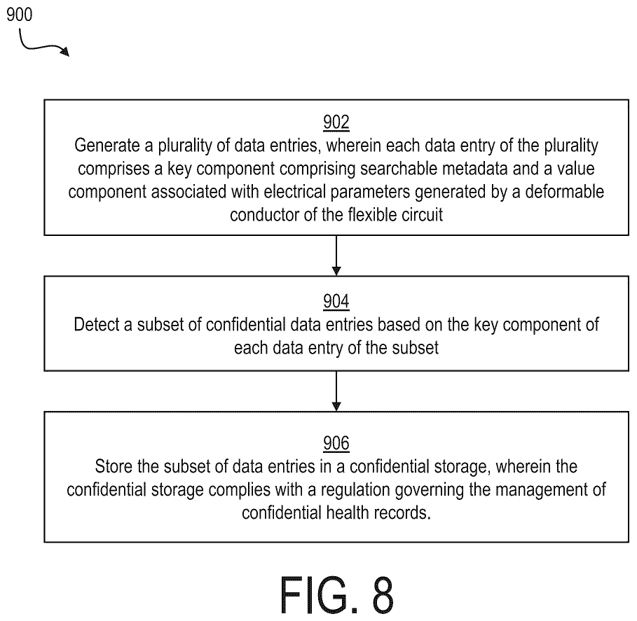

Referring now to FIG. 8, a method 900 of managing health records using a wearable article comprising a flexible circuit is depicted in accordance with at least one non-limiting aspect of the present disclosure. The method 900, for example, can be performed by the system 100 of FIG. 1 or any of its components, including an onboard computing device of the wearable article 104 and/or the stand-alone computing device 106. According to the non-limiting aspect of FIG. 8, the method 900 can include generating 902, via the wearable article, a plurality of data entries. Each data entry of the plurality can include a key component comprising searchable metadata and a value component associated with electrical parameters generated by a deformable conductor of the flexible circuit. For example, the electrical parameters generated by the deformable conductor of the flexible circuit can vary as a user 102 (FIG. 1) moves while wearing the wearable article 104 (FIG. 1). As previously discussed, the electrical parameters can include at least one of an inductance, a resistance, a voltage drop, a capacitance, and an electromagnetic field, or combinations thereof.

In further reference to FIG. 8, the method 900 can further include detecting 904 a subset of confidential data entries based on the key component of each data entry of the subset. For example, the key component can include at least one of a time-stamp, a geo-location stamp, and a device-identifying stamp, or combinations thereof. The method 900 can subsequently include storing 906 the subset of data entries in a confidential storage, wherein the confidential storage complies with a regulation governing the management of confidential health records. For example, the confidential storage can include a non-fungible token hosted in a regulatory compliant way on a blockchain network. However, according to other non-limiting aspects, the confidential storage can include a confidential server on a restricted side of a firewall.

It shall be appreciated that other implementations of data generated by flexible circuits on wearable articles and NFTs can be beneficial for a variety of purposes. For example, conventional blockchains 107 (FIG. 2) generally verify the content of a hosted distributed ledger 210 (FIG. 2) via a proof-of-work or proof-of-stake methodology. However, proof-of-work methodologies generally require each node to solve an increasingly complex cryptographic puzzle, requiring an excessive amount of processing resources and electricity. Obviously, this can have a detrimental impact on the environment and can exclude certain nodes that lack the technical qualifications and resources from participating in the blockchain network 107 (FIG. 2). Alternately, proof-of-stake methodologies require certain participants to stake tokens (e.g., cryptocurrencies) to vet transactions and other content hosted on the distributed ledger 210 (FIG. 2). However, this can lead to centralized power as those with more tokens to stake have more influence over the blockchain network 107 (FIG. 2). Tokens are generally minted by the blockchain network 107 (FIG. 2) when a cryptographic puzzle is solved or the staked transaction is verified. According to the present disclosure, flexible circuits on wearable articles can be implemented to provide an alternate means of verifying the content of a hosted distributed ledger 210 (FIG. 2).

For example, according to some non-limiting aspects, the blockchain network 107 (FIG. 2) can be alternately configured to mint new tokens when the key component 602_{A-n} (FIGS. 6 and 7) and/or value component 604_{A-n} (FIGS. 6 and 7) of a particular data entry 601_{A-n} (FIGS. 6 and 7) meet a predetermined threshold value. For example, the blockchain network 107 (FIG. 2) can be configured to mint a new token when the value component 604_{A-n} (FIGS. 6 and 7) of a particular data entry 601_{A-n} (FIGS. 6 and 7) exceeds a predetermined threshold. In other words, when the user performs a required amount of physical activity, the flexible circuits of the wearable article can generate a certain amount of signals based on the required amount of physical activity. Thus, value component 604_{A-n} (FIGS. 6 and 7) of a particular data entry 601_{A-n} (FIGS. 6 and 7) will contain data associated with the certain amount of signals, and the blockchain network 107 (FIG. 2) can thus determine that the user performed the required amount of physical activity based on the value component 604_{A-n}. As such, the blockchain network 107 (FIG. 2) can reward the user (or the community, at large) for performing the required amount of physical activity by minting a new token.

According to some non-limiting aspects, the blockchain network 107 (FIG. 2) can use the relative sizes of the key component 602_{A-n} (FIGS. 6 and 7) and value component 604_{A-n} (FIGS. 6 and 7) of a particular data entry 601_{A-n} (FIGS. 6 and 7) to determine whether or not to mint a new token. For example, if the value component 604_{A-n} of the data entry 601_{A-n} includes a large quantity of data and the key component 602_{A-n} of the data entry 601_{A-n} includes a small quantity of data, it can indicate that the user was extremely active over a short period of time. Thus, the blockchain network 107 (FIG. 2) can reward the user (or the community, at large) for performing a required amount of physical activity within a predetermined period of time by minting a new token. It shall be appreciated that such non-limiting aspects can be particularly beneficial for user's attempting to track and/or incentivize the physical activity of a user. For example, insurance companies and/or corporations may use such non-limiting aspects to incentivize insured individual and/or employees to be more active. Thus, the minted tokens can be used to subsidize and/or reduce premiums, fund Health Savings Accounts, or can be applied to pay for medical expenses.

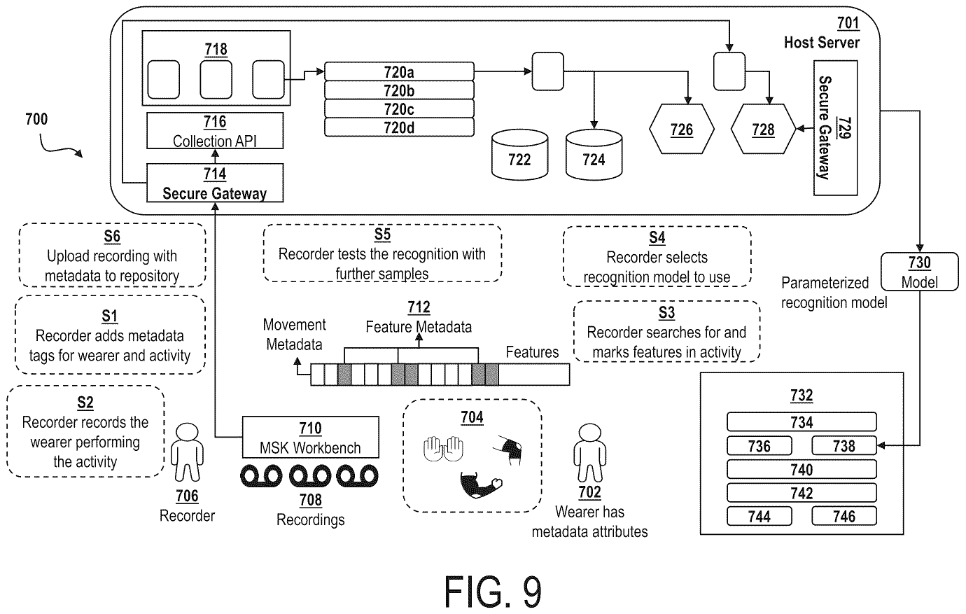

Referring now to FIG. 9, an architecture 700 that can be deployed via the system 100 of FIG. 1 to use a wearable article featuring flexible circuits in conjunction with a non-fungible token NFT hosted on a blockchain, in accordance with at least one non-limiting aspect of the present disclosure. According to the non-limiting aspect, the architecture 700 can include a wearable article 704 worn by a user 702, a workbench worked by a recorder 706, a host server 701, and a data repository 732, or library. For example, the wearable article 704 can include any of the devices employing flexible circuitry disclosed herein, including those incorporated by reference, such as a glove, a brace, a sleeve, a shirt, a hat, pants, a wrap, and/or socks or shoes. The workbench 710, for example, can include any fully managed service that enables the recorder 706 to build and run applications to process streaming data, such as Apache Kafka (e.g., Amazon's Managed Streaming for Apache Kafka, or MSK, etc.). The workbench 710 can be configured to identify features used for recognition and associate tags within a dataset 712. Under some circumstances, a clinc product, such as MSK-clinic can be used.

The architecture 700 of FIG. 9 can enable specific functionality associated with the generation of data via the wearable articles 704 to mint vian NFTs. For example, the recorder 706 can add S1 metadata for tags for the wearer 702 and the desired activity via the workbench 710. The recorder 706 can then record S2 the wearer performing the activity, which generates data associated with electrical parameters and signals produced via the flexible circuits of the wearable article 704 and specifically, the deformable conductor of the flexible circuits. This results in at least one recording 708 featuring a dataset 712 that can include features, movement metadata, and feature metadata. It shall be appreciated that such features of the dataset 712 can constitute any of the key components 602_{A-n} or value components 604_{A-n} of the data entries 601_{A-n} of the table 600 of FIGS. 6 and 7. The recorder 706 can then search S3 for and mark features in the dataset 712 and, based on the selected features, can select S4 a recognition model to use. The recorder 706 may test S5 the recognition model and then upload S6 the recording 708 to the host server 701 via the workbench 710.

Still referring to FIG. 9, the host server 701 can be configured to process and mint the dataset 712 into an NFT. According to some non-limiting aspects, the host server 701 can be located in the cloud and can include one or more servers operated by one or more entities. According to the non-limiting aspect of FIG. 9, the host server 701 can include a secure gateway 714, an interoperability API 716, and one or more harvesting modules 718 designed to assess the dataset 712 of the recordings 708 and ensure they are properly processed for storage, for example, via the one or more repositories 109 (FIG. 1) in accordance with the predefined one or more rules. The harvesting modules 718 can transmit the dataset 712 to one or more third-party datastores 720_{a-d}, including those sourced from a third-party database. Portions of the dataset 712 can be stored in a non-anonymous datastore 722 for personal and/or recreational uses, an anonymous datastore 724 for private or medical purposes, or can be minted into an NFT and stored in a blockchain 726. A second secure gateway 729, based on harvested features form the dataset 712, can ensure a proper model 728 is applied to the dataset 712 to promote interoperability with an avatar or environment. The gateway 729, for example, can apply a parameterized recognition model 730 to process the dataset 712 from the recordings 708 and transmit it to the data repository 732, which can include an MSK edge library, for example. Here, the dataset 712 can be stored in accordance to various parameters (identified via the applied metatags), including wearer identify 734, kinematics 736, recognition 738, data session/pose/recording 740, data management and or miscellaneous tags 742, connections 744, and/or sensors 746.

As such, it shall be appreciated that the architecture 700 of FIG. 9 enables the system 100 of FIG. 1 to disposition data generated by the wearable article 704 to store vast amounts of structured and unstructured data at scale in an original, raw format, via one or more repositories 109 (FIG. 1) in accordance with the predefined rules. It employs a standardized taxonomy of metadata tags that can be used to annotate recordings 708 and datasets 712 based on movement/pose categories to enable feature recognition and proper interoperability with avatars regardless of source. The taxonomy, for example, can be extended to sub-segments or features based on the metatags within a recording 708. Conventional devices, systems, and methods, are less efficient because desired movement entries are difficult to separate from undesired data associated with non-movements. The segment/tagging of datasets 712 provided by the architecture 700 of FIG. 9 enables undesired data to be separated from and discarded, thereby enhancing the precision of replicated motions. The data repository 732 can be actively curated over time to ensure that the best data is used for movement replication. In other words, curation can be employed to continually improve the motion recognition model. According to some non-limiting aspects, curation can be accelerated via the provision test sets of data, such that the model is trained.

Since the inventive principles of this patent disclosure can be modified in arrangement and detail without departing from the inventive concepts, such changes and modifications are considered to fall within the scope of the following claims. The use of terms such as first and second are for purposes of differentiating different components and do not necessarily imply the presence of more than one component.

Various aspects of the subject matter described herein are set out in the following numbered clauses:

Clause 1. A computer-implemented method of autonomously dispositioning data generated by a wearable article in compliance with multiple application-specific requirements, the method including predefining, via a processor, one or more rules by which data generated by a wearable article should be managed, wherein the one or more rules include definition of a triggering event, receiving, via the processor, data associated with motions of the wearable article, wherein the data includes information associated with electrical parameters generated by the wearable article that vary with the motions of the wearable article, detecting, via the processor, an initiation of the triggering event, and managing, via the processor, subsequent data generated by the wearable article, including data associated with varying electrical parameters, in accordance with the predefined one or more rules.

Clause 2. The computer-implemented method according to clause 1, wherein managing the subsequent data generated by the wearable article further includes transmitting, via the processor, the subsequent data generated by the wearable article to a first repository of a plurality of repositories.

Clause 3. The computer-implemented method according to either of clauses 1 or 2, wherein the triggering event includes a motion performed by a user of the wearable article, and wherein the method further includes correlating, via the processor, the electrical parameters generated by the wearable article to physical parameters associated with one or more portions of the wearable article, and determining, via the processor, that the user of the wearable article has performed the motion based on the correlation.

Clause 4. The computer-implemented method according to any of clauses 1-3, wherein the motion performed by the user of the wearable article includes a personal motion, and wherein the first repository of the plurality of repositories includes a personal server.

Clause 5. The computer-implemented method according to any of clauses 1-4, wherein the motion performed by the user of the wearable article includes a medical motion, and wherein the first repository of the plurality of repositories includes a confidential storage on a restricted side of a firewall.

Clause 6. The computer-implemented method according to any of clauses 1-5, wherein the motion performed by the user of the wearable article includes a motion of interest to an insurance company associated with the user of the wearable article, and wherein the first repository of the plurality of repositories includes a server associated with the insurance company.

Clause 7. The computer-implemented method according to any of clauses 1-6, wherein managing the subsequent data generated by the wearable article further includes generating, via the processor, a non-fungible token associated with the subsequent data generated by the wearable article, and storing, via the processor, the non-fungible token on a blockchain network.

Clause 8. The computer-implemented method according to any of clauses 1-7, wherein the predefining the one or more rules further includes programming, via the processor, the one or more rules into a smart contract executed by the blockchain network.

Clause 9. The computer-implemented method according to any of clauses 1-8, wherein the triggering event includes an engagement with a button associated with the wearable article.

Clause 10. The computer-implemented method according to any of clauses 1-9, wherein the button is physically positioned on the wearable article.

Clause 11. The computer-implemented method according to any of clauses 1-10, wherein the button is virtually presented via a display of a computing device communicably coupled to the wearable article.

Clause 12. The computer-implemented method according to any of clauses 1-11, wherein detecting the initiation of the triggering event further includes detecting, via the processor, that an ancillary device has been activated.

Clause 13. A system, including a wearable article including a flexible circuit, wherein the flexible circuit includes a trace made from a deformable conductor configured to generate varying electrical parameters in response to motions of the wearable article, and a computing device communicably coupled to the wearable article, wherein the computing device includes a processor and a memory configured to store instructions that, when executed by the processor, cause the computing device to predefine one or more rules by which data generated by a wearable article should be managed based on a user input, wherein the one or more rules include definition of a triggering event, receive data associated with motions of the wearable article, wherein the data includes information associated with the varying electrical parameters generated by the deformable conductor, detect an initiation of the triggering event, and manage subsequent data generated by the wearable article, including data associated with varying electrical parameters, in accordance with the predefined one or more rules.

Clause 14. The system according to clause 13, wherein managing the subsequent data generated by the wearable article further includes transmitting the subsequent data generated by the wearable article to a first repository of a plurality of repositories.

Clause 15. The system according to either of clauses 13 or 14, wherein the triggering event includes a motion performed by a user of the wearable article, and wherein, when executed by the processor, the instructions further cause the computing device to correlate the electrical parameters generated by the wearable article to physical parameters associated with one or more portions of the wearable article, and determine that the user of the wearable article has performed the motion based on the correlation.

Clause 16. The system according to any of clauses 13-15, wherein the motion performed by the user of the wearable article includes a personal motion, and wherein the first repository of the plurality of repositories includes a personal server.

Clause 17. The system according to any of clauses 13-16, wherein the motion performed by the user of the wearable article includes a medical motion, and wherein the first repository of the plurality of repositories includes a confidential storage on a restricted side of a firewall.

Clause 18. The system according to any of clauses 13-17, wherein managing the subsequent data generated by the wearable article further includes generating, via the processor, a non-fungible token associated with the subsequent data generated by the wearable article, and storing, via the processor, the non-fungible token on a blockchain network.

Clause 19. A method of managing health records using a wearable article including a flexible circuit, the method including generating, via the wearable article, a plurality of data entries, wherein each data entry of the plurality includes a key component including searchable metadata and a value component associated with electrical parameters generated by a deformable conductor of the flexible circuit, detecting, via a computing device, a subset of confidential data entries based on the key component of each data entry of the subset, and storing, via the computing device, the subset of data entries in a confidential storage, wherein the confidential storage complies with a regulation governing the management of confidential health records.

Clause 20. The method according to clause 19, wherein the confidential storage is a non-fungible token hosted on a blockchain network.

Clause 21. The method according to either of clauses 19 or 20, wherein the confidential storage is a confidential server on a restricted side of a firewall.

Clause 22. The method according to any of clauses 19-21, wherein the key component includes at least one of a time-stamp, a geo-location stamp, and a device-identifying stamp, or combinations thereof.

Clause 23. The method according to any of clauses 19-22, wherein the electrical parameters generated by the deformable conductor of the flexible circuit vary as a user moves while wearing the wearable article.

Clause 24. The method according to any of clauses 19-23, wherein the electrical parameters include at least one of an inductance, a resistance, a voltage drop, a capacitance, and an electromagnetic field, or combinations thereof.

All patents, patent applications, publications, or other disclosure material mentioned herein, are hereby incorporated by reference in their entirety as if each individual reference was expressly incorporated by reference respectively. All references, and any material, or portion thereof, that are said to be incorporated by reference herein are incorporated herein only to the extent that the incorporated material does not conflict with existing definitions, statements, or other disclosure material set forth in this disclosure. As such, and to the extent necessary, the disclosure as set forth herein supersedes any conflicting material incorporated herein by reference and the disclosure expressly set forth in the present application controls.

The present invention has been described with reference to various exemplary and illustrative aspects. The aspects described herein are understood as providing illustrative features of varying detail of various aspects of the disclosed invention; and therefore, unless otherwise specified, it is to be understood that, to the extent possible, one or more features, elements, components, constituents, ingredients, structures, modules, and/or aspects of the disclosed aspects may be combined, separated, interchanged, and/or rearranged with or relative to one or more other features, elements, components, constituents, ingredients, structures, modules, and/or aspects of the disclosed aspects without departing from the scope of the disclosed invention. Accordingly, it will be recognized by persons having ordinary skill in the art that various substitutions, modifications or combinations of any of the exemplary aspects may be made without departing from the scope of the invention. In addition, persons skilled in the art will recognize, or be able to ascertain using no more than routine experimentation, many equivalents to the various aspects of the invention described herein upon review of this specification. Thus, the invention is not limited by the description of the various aspects, but rather by the claims.

Those skilled in the art will recognize that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although claim recitations are presented in a sequence(s), it should be understood that the various operations may be performed in other orders than those which are described, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

It is worthy to note that any reference to “one aspect,” “an aspect,” “an exemplification,” “one exemplification,” and the like means that a particular feature, structure, or characteristic described in connection with the aspect is included in at least one aspect. Thus, appearances of the phrases “in one aspect,” “in an aspect,” “in an exemplification,” and “in one exemplification” in various places throughout the specification are not necessarily all referring to the same aspect. Furthermore, the particular features, structures or characteristics may be combined in any suitable manner in one or more aspects.

As used herein, the singular form of “a”, “an”, and “the” include the plural references unless the context clearly dictates otherwise.

Directional phrases used herein, such as, for example and without limitation, top, bottom, left, right, lower, upper, front, back, and variations thereof, shall relate to the orientation of the elements shown in the accompanying drawing and are not limiting upon the claims unless otherwise expressly stated.

The terms “about” or “approximately” as used in the present disclosure, unless otherwise specified, means an acceptable error for a particular value as determined by one of ordinary skill in the art, which depends in part on how the value is measured or determined. In certain aspects, the term “about” or “approximately” means within 1, 2,3, or 4 standard deviations. In certain aspects, the term “about” or “approximately” means within 50%, 200%, 105%, 100%, 9%, 8%, 7%, 6%, 5%, 4%, 3%, 2%, 1%, 0.5%, or 0.05% of a given value or range.

In this specification, unless otherwise indicated, all numerical parameters are to be understood as being prefaced and modified in all instances by the term “about,” in which the numerical parameters possess the inherent variability characteristic of the underlying measurement techniques used to determine the numerical value of the parameter. At the very least, and not as an attempt to limit the application of the doctrine of equivalents to the scope of the claims, each numerical parameter described herein should at least be construed in light of the number of reported significant digits and by applying ordinary rounding techniques.

Any numerical range recited herein includes all sub-ranges subsumed within the recited range. For example, a range of “1 to 100” includes all sub-ranges between (and including) the recited minimum value of 1 and the recited maximum value of 100, that is, having a minimum value equal to or greater than 1 and a maximum value equal to or less than 100. Also, all ranges recited herein are inclusive of the end points of the recited ranges. For example, a range of “1 to 100” includes the end points 1 and 100. Any maximum numerical limitation recited in this specification is intended to include all lower numerical limitations subsumed therein, and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited. All such ranges are inherently described in this specification.

Any patent application, patent, non-patent publication, or other disclosure material referred to in this specification and/or listed in any Application Data Sheet is incorporated by reference herein, to the extent that the incorporated materials is not inconsistent herewith. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a system that “comprises,” “has,” “includes” or “contains” one or more elements possesses those one or more elements, but is not limited to possessing only those one or more elements. Likewise, an element of a system, device, or apparatus that “comprises,” “has,” “includes” or “contains” one or more features possesses those one or more features, but is not limited to possessing only those one or more features.

Instructions used to program logic to perform various disclosed aspects can be stored within a memory in the system, such as dynamic random access memory (DRAM), cache, flash memory, or other storage. Furthermore, the instructions can be distributed via a network or by way of other computer readable media. Thus a machine-readable medium may include any mechanism for storing or transmitting information in a form readable by a machine (e.g., a computer), but is not limited to, floppy diskettes, optical disks, compact disc, read-only memory (CD-ROMs), and magneto-optical disks, read-only memory (ROMs), random access memory (RAM), erasable programmable read-only memory (EPROM), electrically erasable programmable read-only memory (EEPROM), magnetic or optical cards, flash memory, or a tangible, machine-readable storage used in the transmission of information over the Internet via electrical, optical, acoustical or other forms of propagated signals (e.g., carrier waves, infrared signals, digital signals, etc.). Accordingly, the non-transitory computer-readable medium includes any type of tangible machine-readable medium suitable for storing or transmitting electronic instructions or information in a form readable by a machine (e.g., a computer). specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

As used in any aspect herein, any reference to a processor or microprocessor can be substituted for any “control circuit,” which may refer to, for example, hardwired circuitry, programmable circuitry (e.g., a computer processor including one or more individual instruction processing cores, processing unit, processor, microcontroller, microcontroller unit, controller, digital signal processor (DSP), programmable logic device (PLD), programmable logic array (PLA), or field programmable gate array (FPGA)), state machine circuitry, firmware that stores instructions executed by programmable circuitry, and any combination thereof. The control circuit may, collectively or individually, be embodied as circuitry that forms part of a larger system, for example, an integrated circuit (IC), an application-specific integrated circuit (ASIC), a system on-chip (SoC), desktop computers, laptop computers, tablet computers, servers, smart phones, etc. Accordingly, as used herein “control circuit” includes, but is not limited to, electrical circuitry having at least one discrete electrical circuit, electrical circuitry having at least one integrated circuit, electrical circuitry having at least one application.

As used in any aspect herein, the term “logic” may refer to an app, software, firmware and/or circuitry configured to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices.

As used in any aspect herein, the terms “component,” “system,” “module” and the like can refer to a computer-related entity, either hardware, a combination of hardware and software, software, or software in execution.

Unless specifically stated otherwise as apparent from the foregoing disclosure, it is appreciated that, throughout the foregoing disclosure, discussions using terms such as “processing,” “computing,” “calculating,” “determining,” “displaying,” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

One or more components may be referred to herein as “configured to,” “configurable to,” “operable/operative to,” “adapted/adaptable,” “able to,” “conformable/conformed to,” etc. Those skilled in the art will recognize that “configured to” can generally encompass active-state components and/or inactive-state components and/or standby-state components, unless context requires otherwise.

What is claimed is:

1. A computer-implemented method of autonomously dispositioning data generated by a wearable article in compliance with multiple application-specific requirements, the method comprising:
predefining, via a processor, one or more rules by which data generated by a wearable article should be managed, wherein the one or more rules comprise definition of a triggering event;
receiving, via the processor, data associated with motions of the wearable article, wherein the data comprises information associated with electrical parameters generated by the wearable article that vary with the motions of the wearable article;
detecting, via the processor, an initiation of the triggering event; and
managing, via the processor, subsequent data generated by the wearable article, including data associated with varying electrical parameters, in accordance with the predefined one or more rules.

2. The computer-implemented method of claim 1, wherein managing the subsequent data generated by the wearable article further comprises transmitting, via the processor, the subsequent data generated by the wearable article to a first repository of a plurality of repositories.

3. The computer-implemented method of claim 2, wherein the triggering event comprises a motion performed by a user of the wearable article, and wherein the method further comprises:
correlating, via the processor, the electrical parameters generated by the wearable article to physical parameters associated with one or more portions of the wearable article; and
determining, via the processor, that the user of the wearable article has performed the motion based on the correlation.

4. The computer-implemented method of claim 3, wherein the motion performed by the user of the wearable article comprises a personal motion, and wherein the first repository of the plurality of repositories comprises a personal server.

5. The computer-implemented method of claim 3, wherein the motion performed by the user of the wearable article comprises a medical motion, and wherein the first repository of the plurality of repositories comprises a confidential storage on a restricted side of a firewall.

6. The computer-implemented method of claim 3, wherein the motion performed by the user of the wearable article comprises a motion of interest to an insurance company associated with the user of the wearable article, and wherein the first repository of the plurality of repositories comprises a server associated with the insurance company.

7. The computer-implemented method of claim 1, wherein managing the subsequent data generated by the wearable article further comprises:
generating, via the processor, a non-fungible token associated with the subsequent data generated by the wearable article; and
storing, via the processor, the non-fungible token on a blockchain network.

8. The computer-implemented method of claim 7, wherein the predefining the one or more rules further comprises programming, via the processor, the one or more rules into a smart contract executed by the blockchain network.

9. The computer-implemented method of claim 1, wherein the triggering event comprises an engagement with a button associated with the wearable article.

10. The computer-implemented method of claim 9, wherein the button is physically positioned on the wearable article.

11. The computer-implemented method of claim 9, wherein the button is virtually presented via a display of a computing device communicably coupled to the wearable article.

12. The computer-implemented method of claim 1, wherein detecting the initiation of the triggering event further comprises detecting, via the processor, that an ancillary device has been activated.

13. A system, comprising:
a wearable article comprising a flexible circuit, wherein the flexible circuit comprises a trace made from a deformable conductor configured to generate varying electrical parameters in response to motions of the wearable article; and
a computing device communicably coupled to the wearable article, wherein the computing device comprises a processor and a memory configured to store instructions that, when executed by the processor, cause the computing device to:
predefine one or more rules by which data generated by a wearable article should be managed based on a user input, wherein the one or more rules comprise definition of a triggering event;
receive data associated with motions of the wearable article, wherein the data comprises information associated with the varying electrical parameters generated by the deformable conductor;
detect an initiation of the triggering event; and
manage subsequent data generated by the wearable article, including data associated with varying electrical parameters, in accordance with the predefined one or more rules.

14. The system of claim 13, wherein managing the subsequent data generated by the wearable article further comprises transmitting the subsequent data generated by the wearable article to a first repository of a plurality of repositories.

15. The system of claim 14, wherein the triggering event comprises a motion performed by a user of the wearable article, and wherein, when executed by the processor, the instructions further cause the computing device to:
correlate the electrical parameters generated by the wearable article to physical parameters associated with one or more portions of the wearable article; and
determine that the user of the wearable article has performed the motion based on the correlation.

16. The system of claim 15, wherein the motion performed by the user of the wearable article comprises a personal motion, and wherein the first repository of the plurality of repositories comprises a personal server.

17. The system of claim 15, wherein the motion performed by the user of the wearable article comprises a medical motion, and wherein the first repository of the plurality of repositories comprises a confidential storage on a restricted side of a firewall.

18. The system of claim 13, wherein managing the subsequent data generated by the wearable article further comprises:
generating, via the processor, a non-fungible token associated with the subsequent data generated by the wearable article; and
storing, via the processor, the non-fungible token on a blockchain network.

19. A method of managing health records using a wearable article comprising a flexible circuit, the method comprising:
generating, via the wearable article, a plurality of data entries, wherein each data entry of the plurality comprises a key component comprising searchable metadata and a value component associated with electrical parameters generated by a deformable conductor of the flexible circuit;
detecting, via a computing device, a subset of confidential data entries based on the key component of each data entry of the subset; and
storing, via the computing device, the subset of data entries in a confidential storage, wherein the confidential storage complies with a regulation governing the management of confidential health records.

20. The method of claim 19, wherein the confidential storage is a non-fungible token hosted on a blockchain network.
